(19) United States
(12) Patent Application Publication
(10) Pub. No.: US 2025/0196465 A1
(43) Pub. Date: Jun. 19, 2025

(54) COMPOSITE SHEET MATERIAL FOR USE AS ACQUISITION DISTRIBUTION LAYER IN AN ABSORBENT ARTICLE

(71) Applicant: Fitesa Film Products LLC, Simpsonville, SC (US)

(72) Inventors: Phillip Mellor, Terre Haute, IN (US); Andrew D. Maschino, Paris, IL (US)

(73) Assignee: Fitesa Film Products LLC, Simpsonville, SC (US)

(21) Appl. No.: 18/980,036

(22) Filed: Dec. 13, 2024

Related U.S. Application Data
(60) Provisional application No. 63/609,505, filed on Dec. 13, 2023.
(60) Provisional application No. 63/730,276, filed on Dec. 10, 2024.

(51) Int. Cl. B32B 3/26; B32B 5/02; B32B 7/12; B32B 27/12; B32B 27/32; B32B 37/12

(52) U.S. Cl. CPC B32B 3/266; B32B 5/022; B32B 7/12; B32B 27/12; B32B 27/32; B32B 37/1284; B32B 2262/0269; B32B 2307/718; B32B 2307/72; B32B 2555/02

(57) ABSTRACT

A composite sheet material having improved fluid management properties is provided. The composite sheet material includes a nonwoven substrate comprising a carded nonwoven having a basis weight greater than about 16 gsm, or a high loft spunbond nonwoven and a film layer having a plurality of apertured protuberances extending outwardly from a face of the film. The composite sheet material is particularly useful in the preparation of absorbent articles.

### CROSS-REFERENCE TO RELATED APPLICATIONS

The present application claims priority of U.S. Provisional Application No. 63/609,505, filed Dec. 13, 2023 and U.S. Provisional Application No. 63/730,276, filed Dec. 10, 2024, the contents of which are hereby incorporated by reference.

### FIELD

The presently-disclosed invention relates generally to nonwoven fabrics, and more particularly to a composite sheet material for use as an acquisition-distribution layer in an absorbent article.

### BACKGROUND

Nonwoven fabrics are used in a variety of applications such as garments, disposable medical products, and absorbent articles such as diapers and personal hygiene products, among others. New products being developed for these applications have demanding performance requirements, including comfort, conformability to the body, freedom of body movement, good softness and drape, adequate tensile strength and durability, and resistance to surface abrasion, pilling or fuzzing. Accordingly, the nonwoven fabrics which are used in these types of products must be engineered to meet these performance requirements.

Typical absorbent articles may include a multilayer construction having an inner layer (also referred to as a top sheet) defining an inner surface that is in contact with the skin of the wearer, an acquisition/distribution layer (also referred to as an ADL component) disposed underlying the top sheet, an absorbent layer comprising a material selected to absorb fluids, and an outer layer (also referred to as a back sheet) defining an outer surface of the article. Typically. the back sheet comprises a material that is impervious to fluids so that any fluids absorbed within the absorbent core do not escape or leak.

Materials typically used in the ADL are typically selected to rapidly transport fluids from the top sheet and into the absorbent core. This rapid transport (also referred to herein as flash permeation) transports the fluid in the z-direction from the top sheet to the absorbent core. In order to prevent fluid from pooling or remaining near the skin of the wearer, it is important that the distribution layer prevents or reduces reverse osmosis of fluids from the absorbent core and back through the top sheet.

Despite significant efforts in developing nonwoven fabrics, there is still a need for products exhibiting improvements in fluid transport without sacrificing other beneficial properties such as tensile strength, flexibility, and elongation.

### SUMMARY

One or more embodiments of the invention may provide a composite sheet material having desirable properties with respect to fluid transport, comfort, softness, and drape while maintaining good mechanical properties, such as tensile strength and elongation. In particular, embodiments of the present invention provide a composite sheet that is particularly useful as a fluid ADL component in absorbent articles. In one embodiment, a composite sheet is provided in which the composite sheet comprises a nonwoven substrate layer comprising a carded nonwoven fabric or a high loft spunbond nonwoven and a microperforated film overlying the nonwoven substrate layer.

In certain embodiments, a composite sheet material is provided in which the composite sheet material comprises a nonwoven substrate and a microperforated film layer overlying the nonwoven substrate. The nonwoven substrate may be selected from the group consisting of a a) carded nonwoven comprising a plurality of staple fibers bonded to each other to form a coherent web, and having a basis weight from about 16 to 80 gsm, and b) a high loft spunbond nonwoven having a density of less than 0.080 g/cm³.

In certain embodiments, the microperforated film layer is bonded to the nonwoven substrate. Typically, the film comprises a plurality of apertured protuberances extending outwardly from a face of the film, the apertures including a first opening disposed opposite the nonwoven substrate, a second opening at a vertex of the aperture, and a continuous sidewall extending between the first and second openings.

In certain embodiments, the plurality of apertured protuberances extend outwardly from a face of the film towards a surface of the nonwoven substrate. In

In certain embodiments, the plurality of apertured protuberances extend outwardly from a face of the film away from a surface of the nonwoven substrate.

In certain embodiments, the nonwoven substrate comprises a through air bonded fabric.

In certain embodiments, the nonwoven substrate comprises a high loft spunbond nonwoven.

In certain embodiments, the nonwoven substrate comprises a resin-bonded nonwoven.

In certain embodiments, a polymer defining the microperforated film layer is contacted with a surface of the nonwoven substrate in a molten or semi-molten state such that the polymer is at least partially integrated into and between the fibers of the nonwoven substrate to form a mechanical bond between the nonwoven substrate and the film layer.

In certain embodiments, the plurality of apertured protuberances have a conical shape.

In certain embodiments, the microperforated film layer comprises polyethylene.

In certain embodiments, the nonwoven substrate comprises a carded nonwoven fabric layer having a basis weight from about 30 to 50 gsm.

In certain embodiments, the high loft spunbond nonwoven is selected from the group consisting of a through air bonded nonwoven, a spunbond nonwoven comprising a plurality of crimped filaments; a spunbond nonwoven in which fibers of the nonwoven comprise a blend of a polymer and a high loft additive, and combinations thereof.

In certain embodiments, the composite sheet material exhibits an average fluid acquisition time of less than 5 seconds after a first fluid insult and less than 7 seconds after a second fluid insult.

In certain embodiments, the composite sheet material exhibits an average fluid acquisition times of less than 3 seconds after a first fluid insult and less than 5 seconds after a second fluid insult.

In certain embodiments, the composite sheet material exhibits an average fluid acquisition time following a single fluid insult of less than 1 seconds, and in particular, less than any one or more of less than 0.8 seconds, less than 0.7 seconds, less than 0.6 seconds, less than 0.5 seconds, less than 0.4 seconds, less than 0.3 seconds, and less than 0.25 seconds.

In certain embodiments, the composite sheet material exhibits a single average fluid acquisition time following a single fluid insult from about 0.2 to 3 seconds, and in particular, from about 0.2 to 1 seconds.

In certain embodiments, the composite sheet material exhibits a fluid rewet value following a single fluid insult of less than 1.0 gram (g), less than 0.5 g, less than 0.1 g, and in particular less than 0.08 g, and more particularly, less than 0.07 g.

In certain embodiments, the composite sheet material exhibits a fluid rewet value following a single fluid insult ranging from about 0.05 to 1.0 g, and in particular, from about 0.06 to 0.08 g.

In certain embodiments, the composite sheet material exhibits a fluid acquisition time after a first fluid insult of less than 1 seconds and a rewet of less than 0.15 grams, and in particular, a fluid acquisition time after a first fluid insult of less than 0.5 seconds and a rewet of less than 0.08 grams.

Embodiments of the invention are also directed to the use of the composite sheet material of one of the preceding claims in an absorbent article, such as use in a diaper or a feminine hygiene pad.

Aspects of the invention are also directed to methods of preparing a composite sheet material. In certain embodiments, the method comprising the steps of:
providing a nonwoven substrate, the nonwoven substrate selected from the group consisting of a carded nonwoven fabric and a high loft spunbond nonwoven;
extruding a polymer film onto a cylinder having a forming surface thereon while the polymer film is in a molten or semi-molten state;
forming a plurality of apertured protuberances in the film to form a formed film layer, wherein each apertured protuberance including a proximal end, a distal end, and a continuous sidewall extending therebetween;
contacting a surface of the nonwoven substrate to a surface of the formed film layer; and
bonding the nonwoven substrate to the formed film to form a composite sheet material.

In certain embodiments, the step of contacting the nonwoven substrate and the formed film layer comprises a step of introducing the nonwoven substrate onto the formed film layer while the formed film layer is still on the surface of said cylinder.

In certain embodiments, the step of contacting the nonwoven substrate and the formed film layer is performed after a step of drawing the formed film layer off the cylinder.

In certain embodiments, the step of bonding comprises passing the composite sheet material through a calender.

In certain embodiments, the plurality of apertured protuberances extend outwardly from a face of the film towards the surface of the nonwoven substrate.

In certain embodiments, the plurality of apertured protuberances extend outwardly from a face of the film away from the surface of the nonwoven substrate.

In certain embodiments, the polymer defining the film layer is contacted with a surface of the nonwoven substrate in a molten or semi-molten state such that the polymer is at least partially integrated into and between fibers of the nonwoven substrate to form a mechanical bond between the nonwoven substrate and the formed film layer.

In certain embodiments, the cylinder includes a slot extending laterally across its length, and wherein a vacuum is applied through the slot to the film layer overlying the slot.

In certain embodiments of the method, the nonwoven substrate comprises a carded nonwoven comprising a plurality of staple fibers bonded to each other to form a coherent web, the carded nonwoven fabric having a basis weight from about 16 to 80 gsm.

In certain embodiments of the method, the high loft spunbond nonwoven has a density of less than 0.080 g/cm³.

In certain embodiments of the method, the high loft spunbond nonwoven is selected from the group consisting of a through air bonded nonwoven, a spunbond nonwoven comprising a plurality of crimped filaments; a spunbond nonwoven in which fibers of the nonwoven comprise a blend of a polymer and a high loft additive, and combinations thereof.

### BRIEF DESCRIPTION OF THE SEVERAL DRAWINGS

Having thus described the invention in general terms, reference will now be made to the accompanying drawings, which are not necessarily drawn to scale, and wherein:

FIG. 1 illustrates a composite sheet material in accordance with embodiments of the present invention;

FIG. 2 is a cross-sectional view of the composite sheet material taken along line 2-2 of FIG. 1;

FIGS. 3A and 3B are cross-sectional views of a composite sheet material in accordance with at least one embodiment of the invention;

FIG. 4 is a photograph image of a cross section of a composite sheet material in accordance with at least one embodiment of the invention;

FIGS. 5-9 are schematic views of apparatuses for preparing a composite sheet material in accordance with at least one embodiment of the invention;

FIG. 10 illustrate an absorbent article in accordance with one or more embodiments of the present invention;

FIG. 11 illustrates an absorbent article in accordance with one or more embodiments of the present invention;

FIG. 12 illustrates a system for preparing a nonwoven fabric in accordance with at least one embodiment of the present invention;

FIG. 13 illustrates a system for preparing a nonwoven fabric in accordance with at least one embodiment of the present invention; and

FIG. 14 is a schematic illustration of an apparatus for preparing a composite sheet material in accordance with at least one embodiment of the invention.

### DETAILED DESCRIPTION

The invention now will be described more fully hereinafter with reference to the accompanying drawings, in which some, but not all embodiments of the inventions are shown. Indeed, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The terms “first,” “second,” and the like, “primary,” “exemplary,” “secondary,” and the like, do not denote any order, quantity, or importance, but rather are used to distinguish one element from another. Further, the terms “a,” “an,” and “the” do not denote a limitation of quantity, but rather denote the presence of “at least one” of the referenced item.

Each embodiment disclosed herein is contemplated as being applicable to each of the other disclosed embodiments. All combinations and sub-combinations of the various elements described herein are within the scope of the invention.

It is understood that where a parameter range is provided, all integers within that range, and tenths and hundredths thereof, are also provided by the invention. For example, “5-10%” includes 5%, 6%, 7%, 8%, 9%, and 10%; 5.0%, 5.1%, 5.2% . . . 9.8%, 9.9%, and 10.0%; and 5.00%, 5.01%, 5.02% . . . 9.98%, 9.99%, and 10.00%.

As used herein, the terms “about,” “approximately,” and “substantially” in the context of a numerical value or range means ±10% of the numerical value or range recited or claimed, and in particular, encompasses values within a standard margin of error of measurement (e.g., SEM) of a stated value or variations ±0.5%, 1%, 5%, or 10% from a specified value.

For the purposes of the present application, the following terms shall have the following meanings:

The term “fiber” can refer to a fiber of finite length or a filament of infinite length.

As used herein, the term “monocomponent” refers to fibers formed from one polymer or formed from a single blend of polymers. Of course, this does not exclude fibers to which additives have been added for color, anti-static properties, lubrication, hydrophilicity, liquid repellency, etc.

As used herein, the term “staple fiber” refers to fibers of discrete lengths, such as lengths from about 10 to 60 millimeters (mm), and in particular, from about 25 to 50 mm.

As used herein, the term “composite” may refer to a structure comprising two or more layers, such as a film layer and a fiber layer or a plurality of fiber layers joined together. The two layers of a composite structure may be joined together such that a substantial portion of their common X-Y plane interface, according to certain embodiments of the invention.

As used herein, the term “multicomponent” refers to fibers formed from at least two polymers (e.g., bicomponent fibers) that are extruded from separate extruders. The at least two polymers can each independently be the same or different from each other, or be a blend of polymers. The polymers are arranged in substantially constantly positioned distinct zones across the cross-section of the fibers. The components may be arranged in any desired configuration, such as sheath-core, side-by-side, pie, island-in-the-sea, and so forth. Various methods for forming multicomponent fibers are described in U.S. Pat. No. 4,789,592 to Taniguchi et al. and U.S. Pat. No. 5,336,552 to Strack et al., U.S. Pat. No. 5,108,820 to Kaneko, et al., U.S. Pat. No. 4,795,668 to Kruege, et al., U.S. Pat. No. 5,382,400 to Pike, et al., U.S. Pat. No. 5,336,552 to Strack, et al., and U.S. Pat. No. 6,200,669 to Marmon, et al., which are incorporated herein in their entirety by reference. Multicomponent fibers having various irregular shapes may also be formed, such as described in U.S. Pat. No. 5,277,976 to Hogle, et al., U.S. Pat. No. 5,162,074 to Hills, U.S. Pat. No. 5,466,410 to Hills, U.S. Pat. No. 5,069,970 to Largman, et al., and U.S. Pat. No. 5,057,368 to Largman, et al., which are incorporated herein in their entirety by reference.

As used herein the terms “nonwoven,” “nonwoven web” and “nonwoven fabric” refer to a structure or a web of material which has been formed without use of weaving or knitting processes to produce a structure of individual fibers or threads which are intermeshed, but not in an identifiable, repeating manner. Nonwoven webs have been, in the past, formed by a variety of conventional processes such as, for example, meltblown processes, spunbond processes, and staple fiber carding processes.

As used herein, the term “carded fabric” refers to a nonwoven fabric comprising staple fibers that are predominantly aligned and oriented in the machine direction using a carding process. Processes and systems for preparing carded fabrics are disclosed, for example, in U.S. Pat. Nos. 3,145,425 and 5,494,736.

As used herein, the term “machine direction” or “MD” refers to the direction of travel of the nonwoven web during manufacturing.

As used herein, the term “cross direction” or “CD” refers to a direction that is perpendicular to the machine direction and extends laterally across the width of the nonwoven web.

As used herein, the term “spunbond” refers to a process involving extruding a molten thermoplastic material as filaments from a plurality of fine, usually circular, capillaries of a spinneret, with the filaments then being attenuated and drawn mechanically or pneumatically. The filaments are deposited on a collecting surface to form a web of randomly arranged substantially continuous filaments which can thereafter be bonded together to form a coherent nonwoven fabric. The production of spunbond non-woven webs is illustrated in patents such as, for example, U.S. Pat. Nos. 3,338,992; 3,692,613, 3,802,817; 4,405,297 and 5,665,300. In general, these spunbond processes include extruding the filaments from a spinneret, quenching the filaments with a flow of air to hasten the solidification of the molten filaments, attenuating the filaments by applying a draw tension, either by pneumatically entraining the filaments in an air stream or mechanically by wrapping them around mechanical draw rolls, depositing the drawn filaments onto a foraminous collection surface to form a web, and bonding the web of loose filaments into a nonwoven fabric. The bonding can be any thermal or chemical bonding treatment, with thermal point bonding being typical.

As used herein “thermal point bonding” involves passing a material such as one or more webs of fibers to be bonded between a heated calender roll and an anvil roll. The calender roll is typically patterned so that the fabric is bonded in discrete point bond sites rather than being bonded across its entire surface.

As used herein, the terms “through air bonded” or “through air bonding” refers to a type of thermal bonding in which a material to be bonded, such as a web of fibers, is subjected to the application of heated gas, such as air, in which the temperature of the heated gas is above the softening or melting temperature of at least one polymer component of the material being bonded. The heated gas causes the at least one polymer component to soften, and under some circumstances, to become semi-molten such that polymers of adjacent fibers fuse together to form thermal bonds. Air thermal bonding may also involve passing a material through a heated oven.

As used herein, the term “resin bonding” or “resin-bonded” refers to a nonwoven web in which the fibers are bonded with a resin adhesive. For example, a nonwoven web can be bonded by gravure bonding. The gravure system includes a solid roll that is engraved with numerous minute indentations. The roller is partially immersed into a resin bath. As the roll turns, excess resin is removed by a doctor blade, which leaves only the adhesive binder in the roller's indentations. An unbonded web is then squeezed through a nip comprising a gravure roll and rubber roll, which causes resin to penetrate into the nonwoven web. After application of the resin, the nonwoven web is dried and cured to form a coherent nonwoven fabric.

As used herein the term “polymer” generally includes, but is not limited to, homopolymers, copolymers, such as, for example, block, graft, random and alternating copolymers, terpolymers, etc. and blends and modifications thereof. Furthermore, unless otherwise specifically limited, the term “polymer” shall include all possible geometrical configurations of the material, including isotactic, syndiotactic and random symmetries.

Embodiments of the invention are directed to a composite sheet material that is particularly useful in the manufacture of absorbent articles, and in particular, disposable feminine hygiene products, diaper products, and the like. As explained in greater detail below, the composite sheet material comprises a multilayer structure having a microperforated film layer overlying at least one carded nonwoven fabric layer.

### Composite Sheet Material

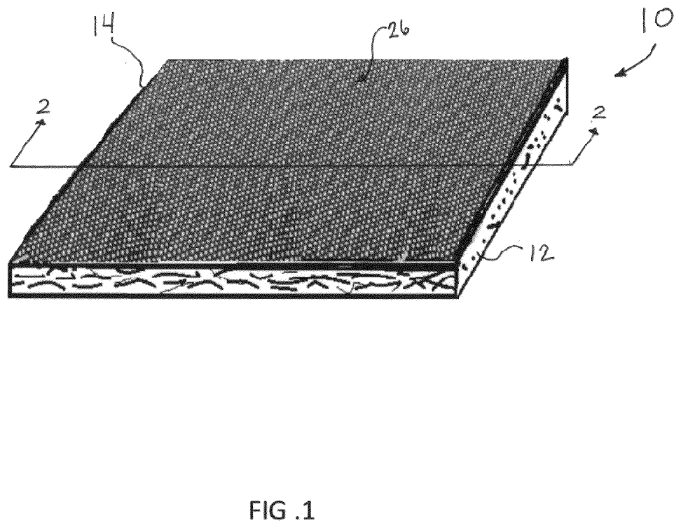

With reference to FIG. 1, a composite sheet material in accordance with at least one embodiment of the invention is shown and generally indicated by reference character 10. In certain embodiments, composite sheet material 10 includes at least one nonwoven substrate layer 12 comprising a plurality of fibers that are bonded to each other to form a coherent web, and a microperforated film layer 14 overlying the nonwoven substrate layer 12. The film layer 14 comprises a plurality of apertured protuberances 26 extending outwardly from a face of the film.

In certain embodiments, the nonwoven substrate layer 12 is selected from the group consisting of a carded nonwoven and a high loft spunbond nonwoven.

As discussed in greater detail below, the apertured protuberances 26 assist in more evenly distributing fluids across the surface of the film layer so that the fluids are not localized in isolated areas and can be quickly transported away from the skin of the wearer.

Generally, the composite sheet 10 may have a basis weight basis weight ranging from about 25 to 160 grams per square meter (gsm), and in particular, from about 30 to 80 gsm, and more particularly, from about 35 to 70 gsm. In a preferred embodiment, the composite sheet has a basis weight that is about 40 to 65 gsm.

The thickness of the composite sheet may range from about 0.5 to 2.5 millimeters (mm), and in particular, from about 0.6 to 1.25 mm, and more particularly, from about 0.6 to 1.0 mm.

### Microperforated Film Layer

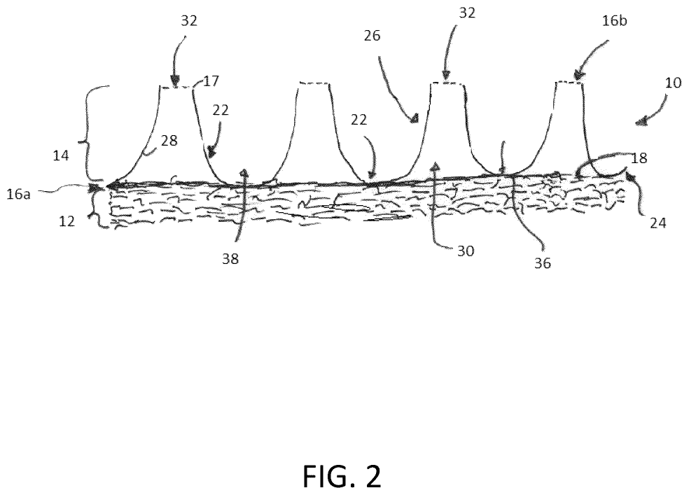

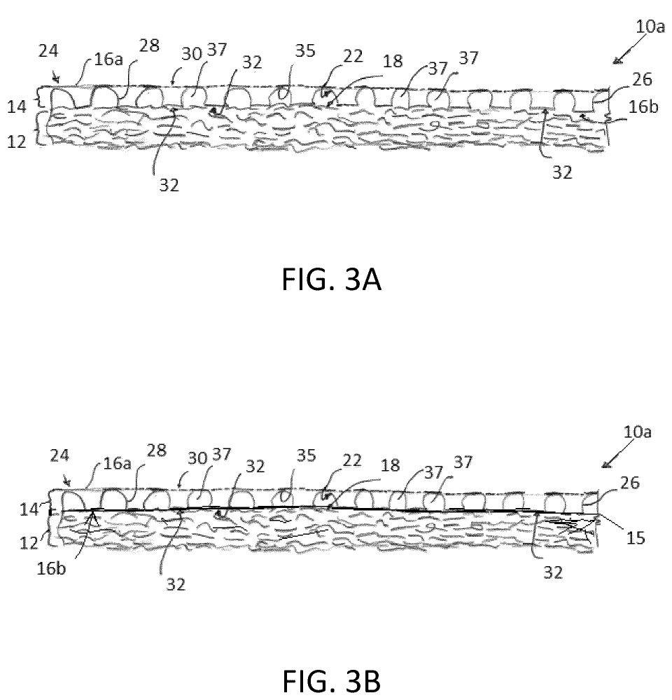

Turning now to FIG. 2 a cross-section views of embodiments of the composite sheet material 10 taken along line 2-2 of FIG. 1 is shown. FIG. 3 illustrates a cross-section view of a further embodiment of a composite sheet 10a. As illustrated in FIGS. 2 and 3, the composite sheet material includes a film layer 14 and a nonwoven substrate layer 12. The film layer 14 includes a first side 22 and a second side 24 that is opposite the first side 22. The film layer 14 comprises a three dimensional topography comprising a plurality of apertured protuberances 26 that extend outwardly from the first side 22 of the film layer 14.

The apertured protuberances 26 each generally include a proximal opening 30, and a corresponding distal opening 32, and a continuous sidewall 28 extending therebetween. In the illustrated embodiment, the distal opening is disposed towards the apex/plateau 17 of the apertured protuberance. As discussed in greater detail below, the apertured openings provide a fluid pathway through the film layer 14 that helps facilitate the movement of fluid from the top sheet and into the underlying nonwoven substrate layer 12. Thereafter, the fluid may be absorbed into an absorbent core (not shown).

In the embodiment illustrated FIG. 2, the second side 24 of the film layer 14 is disposed directly opposite a surface 18 of the nonwoven substrate layer 12. In additional, in this embodiment shown in FIG. 2, the proximal openings 30 are disposed near or adjacent to the proximal face 16a of the film layer 14, and the distal openings 32 are disposed near or adjacent to the distal face 16b of the film layer. The first side 22 of the film layer 14 also includes land areas 34 in between the apertured protuberances 26. The second side 24 of the film layer 14 also includes land areas 36 in between the proximal openings 30.

In the embodiment shown in FIG. 2, the plurality of protuberances 26 also define a plurality of interconnected recesses or valleys 38 that comprise the spaces in between the continuous sidewalls of the plurality of protuberances 26.

In some embodiments, the continuous sidewall 28 of the apertured protuberances 26 may have a generally conical shape in which the cross-sectional diameter of the apertured protuberances narrows as it moves from the proximal to the distal openings. In other words, the proximal openings 30 have may have a larger diameter than the distal openings 32.

In other embodiments, the continuous sidewalls 28 of the apertured protuberances 26 may have other shapes, such as circular, oval, square, rectangular, triangular, non-uniform, or the like. In some embodiments, the cross-section of the apertured protuberances may have a relatively constant diameter between the proximal and distal endings. In other embodiments, the cross-section of the apertured protuberances may have a variable diameter between the proximal and distal endings.

In certain embodiments, the film layer 14 is thermally bonded to the surface 18 of the nonwoven substrate layer 12. In one embodiment, the film layer 14 is attached to the nonwoven substrate layer via thermal bonds at points of contact between landed areas 36 and fibers at the surface 18 of the nonwoven substrate layer. In some embodiments, the polymer from which the film layer is formed is deposited onto surface 18 of the nonwoven substrate layer in a molten or semi-molten state such that the polymer is able to flow between the fibers, and at least some of the fibers are able to embed within the film layer. In this way, the staple fibers are at least partially integrated into film layer to form a mechanical bond between the nonwoven substrate layer and the film layer.

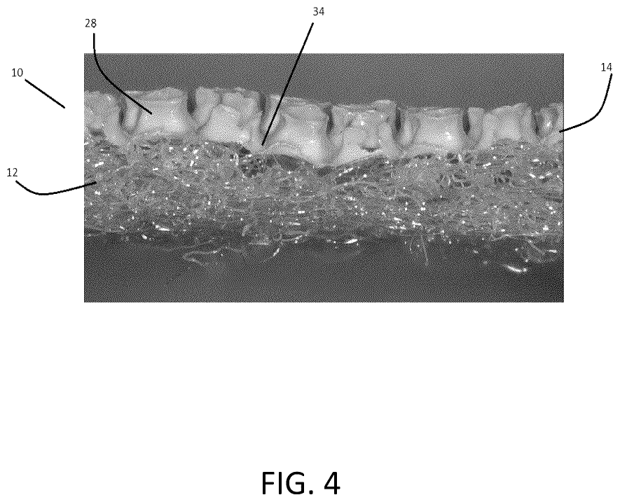

With reference to FIG. 4, a photograph image of a cross section of a composite sheet material in accordance with the embodiment shown in FIG. 2. As shown, the film layer 14 having a plurality of protuberances 26 overlies a nonwoven substrate layer 12. The film layer 14 is joined to the nonwoven substrate layer 12 at landed areas 34.

Referring back to FIG. 3A, an embodiment of the composite sheet material 10a is illustrated in which the film layer 14 is oriented in the opposite direction than the embodiment illustrated in FIG. 2 relative to the nonwoven substrate layer. In this embodiment, the first side 22 of the film layer 14 is disposed opposite surface 18 of the nonwoven substrate layer 12. Accordingly, in comparison to the embodiment of FIG. 2, the plurality of apertured projections extend outwardly from the first side of the film layer 14 towards the surface 18 of the nonwoven substrate layer 12. That is, the film layer 14 is oriented such that the plurality of apertured protuberances 26 extend outwardly from the first side 22 of the film layer 14 in the direction of the nonwoven substrate layer 12, and the second side 24 defines an outer exterior surface of the composite sheet material 10a.

As in the embodiment of FIG. 2, the composite sheet material 10a includes a plurality of proximal openings and distal openings. In this embodiment shown in FIG. 3A, the proximal openings 30 are disposed near or adjacent to the proximal face 16a of the film layer 14, and the distal openings 32 are disposed near or adjacent to the distal face 16b of the film layer.

The first side 22 of the film layer 14 also includes raised land areas 35 in between the apertured protuberances 26 that define a plurality of interconnected confined spaces 37 that are present between the surface 18 of the nonwoven substrate layer 12 and the first side 22 of the film layer 14.

In the embodiment illustrated in FIG. 3A, the film layer 14 may be thermally bonded to the surface 18 of the nonwoven substrate layer 12. In one embodiment, the film layer 14 is attached to the nonwoven substrate layer via thermal bonds at point at regions of contact between the distal face 16b of the film layer and fibers at the surface 18 of the nonwoven substrate layer. In some embodiments, the polymer from which the film layer is formed is deposited onto surface 18 of the nonwoven substrate layer in a molten or semi-molten state such that the polymer is able to flow between the fibers, and at least some of the fibers are able to embed within the film layer. In this way, the fibers of the nonwoven substrate layer are at least partially integrated into film layer to form a mechanical bond between the nonwoven substrate layer and the film layer.

In certain embodiments of the composite sheet material, the film layer 14 may be adhesively bonded to the surface 18 of the nonwoven substrate 16. In this regard, FIG. 3B illustrates an embodiment of the invention in which the composite sheet comprises an adhesive layer 15 that is disposed between the film layer 18 and the nonwoven substrate. The adhesive layer 15 may be continuous or discontinuous.

Suitable adhesives may include clastic adhesives and construction adhesives. Suitable adhesive materials may include thermoplastic elastomers, such as adhesives comprising one or more of styrene isoprene styrene (SIS), styrene butadiene styrene (SBS), styrene ethylene butylene styrene (SEBS) and olefinic based elastomer adhesives. Further examples of suitable adhesives include polyethylene, polypropylene, ethylene vinyl acetate based melt adhesives, and copolymers of such polymers. The amount of adhesive may range from 0.2 weight percent to 2 weight percent, based on an add on weight percent of the total weight of the composite sheet material, and in particular, from about 0.5 to 1.0 weight percent.

The adhesive layer may be applied using any suitable coating method including spray-on methods, use of a doctor blade, gravure roll, printing methods, such as flexographic printing methods, and the like. The adhesive may be applied to a surface of the nonwoven substrate layer, a surface of the film layer, a combination thereof.

In an embodiment, the apertured protuberances 26 may be arranged in a pattern having about 10 to about 60 protuberances per linear inch or “mesh,” i.e., about 10 mesh to about 60 mesh. The pattern may be a hexagonal pattern, a square pattern, a staggered pattern, or any other type of pattern or design. In an embodiment, the apertured protuberances 26 may be arranged in a 10-25 mesh pattern. In an embodiment, the apertured protuberances 26 may be arranged in about an 11 mesh pattern. In an embodiment, the apertured protuberances 26 may be arranged in about a 22 mesh pattern. In an embodiment, the apertured protuberances 26 may be arranged in a 40 mesh pattern. In an embodiment, the proximal apertures 26 may be hexagonal in shape and have approximately the same size. In an embodiment, the proximal apertures 26 may have different sizes and/or shapes.

In certain embodiments, the density of the microperforations may be range from about 250 to 700 microperforations per cm², and in particular, from about 300 to 600 microperforations per cm².

In certain embodiments, the distal openings of the apertured protuberances have average diameters ranging from about 100 to 300 micrometers (μm), and in particular, from about 170 to 285 μm, and more particularly, from about 185 to 240 μm.

The basis weight of the film layer 14 may range from about 5 to 50 micrometers gsm, and in particular, from about 8 to 40 gsm, and more particularly, from about 10 to 30 gsm.

Suitable polymers for the film layer 14 may include one or more polyolefins, including but not limited to polyethylene, ultra-low density polyethylene, low density polyethylene, lincar low density polyethylene, linear medium density polyethylene, high density polyethylene, polypropylene, ethylene-vinyl acetates, metallocene-catalyzed polyolefins, Ziegler-Natta catalyzed polyolefins, as well as other polymers. In a preferred embodiment, the film layer comprises a polyethylene polymer.

Other suitable polymers may include, but are not limited to, elastomeric polymers, including but not limited to polypropylene based elastomers, ethylene based elastomers, copolyester based elastomers, olefin block copolymers, styrenic block copolymers and the like, or combinations thereof.

In some embodiments, the film layer may also include one or more functional additives, such as surfactants, fillers, colorants, opacifying agents, antioxidants, UV protectants/stabilizers and/or other additives known in the art may also be used in the film layer.

### High Loft Spunbond Nonwoven

In certain embodiments, the nonwoven substrate layer comprises a high loft spunbond nonwoven fabric. The high loft spunbond nonwoven fabric may comprise a plurality of continuous crimped filaments, a through air bonded spunbond fabric layer, a nonwoven including a high loft additive, or one or more combinations thereof.

Advantageously, it has been discovered that the use of a high loft spunbond nonwoven helps to improve the fluid management properties of the resulting composite sheet material. The high loft spunbond nonwoven provides improved resiliency, porosity, density, and loft (e.g., thickness) in comparison to a spunbond nonwoven fabric having a conventional loft. In certain embodiments, the high loft spunbond nonwoven is characterized by the absence of thermal point bonding or thermal calender bonding.

In comparison to a conventional loft spunbond nonwoven fabric, a high loft spunbond nonwoven fabric is generally characterized by an increased thickness for a given basis weight. More particularly, high loft nonwovens exhibits increased lofts, lower densities, higher resiliencies, and increased air permeability in comparison to a conventional loft spunbond nonwoven fabric. As used herein, the term “conventional spundbond nonwoven fabric” refers to a spunbond nonwoven fabric that has been subjected to a thermal bonding process or other process that results in compaction and reduction in thickness of the spunbond nonwoven fabric. In addition, conventional nonwoven spunbond fabrics typically exhibit densities greater than 0.080 g/cm³.

Generally, the loft (e.g., thickness) of the high loft spunbond nonwoven fabric layer may be from about 0.25 to 1 mm, and in particular, from about 0.3 to 0.8 mm. In certain preferred embodiments, the loft of the high loft spunbond nonwoven is from about 0.35 to 0.6 mm.

In certain embodiments, the high loft spunbond nonwoven fabric has a density from about 0.02 to 0.08 g/cm³, and in particular, from about 0.045 to 0.065 g/cm³, and more particularly, from about 0.050 to 0.060 g/cm³.

In certain embodiments, the high loft spunbond nonwoven fabric exhibits a density of at least 0.020 g/cm³, at least 0.021 g/cm³, at least 0.022 g/cm³, at least 0.023 g/cm³, at least 0.024 g/cm³, at least 0.025 g/cm³, at least 0.026 g/cm³, at least 0.027 g/cm³, at least 0.028 g/cm³, at least 0.029 g/cm³, at least 0.030 g/cm³, at least 0.031 g/cm³, at least 0.032 g/cm³, at least 0.033 g/cm³, at least 0.034 g/cm³, at least 0.035 g/cm³, at least 0.036 g/cm³, at least 0.037 g/cm³, at least 0.038 g/cm³, at least 0.039 g/cm³, at least 0.040 g/cm³, at least 0.401 g/cm³, at least 0.042 g/cm³, at least 0.043 g/cm³, at least 0.044 g/cm³, at least 0.045 g/cm³, at least 0.046 g/cm³, at least 0.047 g/cm³, at least 0.048 g/cm³, at least 0.049 g/cm³, at least 0.050 g/cm³, at least 0.051 g/cm³, at least 0.052 g/cm³, at least 0.053 g/cm³, at least 0.054 g/cm³, at least 0.055 g/cm³, at least 0.056 g/cm³, at least 0.057 g/cm³, at least 0.058 g/cm³, at least 0.059 g/cm³, at least 0.060 g/cm³, at least 0.061 g/cm³, at least 0.062 g/cm³, at least 0.063 g/cm³, at least 0.064 g/cm³, at least 0.065 g/cm³, at least 0.066 g/cm³, at least 0.067 g/cm³, at least 0.068 g/cm³, at least 0.069 g/cm³, at least 0.070 g/cm³, at least 0.071 g/cm³, at least 0.072 g/cm³, at least 0.073 g/cm³, at least 0.074 g/cm³, at least 0.075 g/cm³, at least 0.076 g/cm³, at least 0.077 g/cm³, at least 0.078 g/cm³, and at least 0.079 g/cm³.

In certain embodiments, the high loft spunbond nonwoven fabric exhibits a density of less than 0.080 g/cm³, less than 0.079 g/cm³, less than 0.078 g/cm³, less than 0.077 g/cm³, less than 0.076 g/cm³, less than 0.075 g/cm³, less than 0.074 g/cm³, less than 0.073 g/cm³, less than 0.072 g/cm³, less than 0.071 g/cm³, less than 0.070 g/cm³, less than 0.069 g/cm³, less than 0.068 g/cm³, less than 0.067 g/cm³, less than 0.066 g/cm³, less than 0.065 g/cm³, less than 0.064 g/cm³, less than 0.063 g/cm³, less than 0.062 g/cm³, less than 0.061 g/cm³, less than 0.060 g/cm³, less than 0.059 g/cm³, less than 0.058 g/cm³, less than 0.057 g/cm³, less than 0.056 g/cm³, less than 0.055 g/cm³, less than 0.054 g/cm³, less than 0.053 g/cm³, less than 0.052 g/cm³, less than 0.051 g/cm³, less than 0.050 g/cm³, less than 0.049 g/cm³, less than 0.048 g/cm³, less than 0.047 g/cm³, less than 0.046 g/cm³, less than 0.045 g/cm³, less than 0.044 g/cm³, less than 0.043 g/cm³, less than 0.042 g/cm³, less than 0.041 g/cm³, less than 0.040 g/cm³, less than 0.039 g/cm³, less than 0.038 g/cm³, less than 0.037 g/cm³, less than 0.036 g/cm³, less than 0.035 g/cm³, less than 0.034 g/cm³, less than 0.033 g/cm³, less than 0.032 g/cm³, less than 0.031 g/cm³, less than 0.030 g/cm³, less than 0.029 g/cm³, less than 0.028 g/cm³, less than 0.027 g/cm³, less than 0.026 g/cm³, less than 0.025 g/cm³, less than 0.024 g/cm³, less than 0.023 g/cm³, less than 0.022 g/cm³, and less than 0.021 g/cm³.

In addition, the high loft nonwoven fabric may exhibit an air permeability of 650 to 1500 cubic feet per minute (cfm), and in particular, from about 750 to 1250 cfm, and more particularly, 950 to 1150 cfm. Unless otherwise stated air permeability is measured in accordance with test method WSP 70.7.

High loft spunbond nonwoven fabrics in accordance with certain embodiments of the invention typically exhibit a percent increase in thickness of at least 20% in comparison to a similar nonwoven fabric which does not comprise crimped filaments or a high loft additive, and in which the similar nonwoven fabric has been subjected to web compaction process, such as a step of thermal calender bonding, such as thermal point bonding. As used herein, the term “similar nonwoven” means 1) the percent difference in basis weights between the high loft nonwoven and the similar nonwoven fabric is less than 2%, and in particular, less than 1%, and more particularly, less than 0.5 percent, and 2) the percent difference in average fiber diameter between the high loft nonwoven and the similar nonwoven fabric is less than 5%, and in particular, less than 4%, and more particularly, less than 3 percent. In certain embodiments, the similar nonwoven fabric may comprises filaments having the identical chemical or polymer composition as the high loft nonwoven fabric.

In accordance with certain embodiments, the basis weight of the high loft nonwoven fabric may vary depending on the end use application of the fabric. For example, in embodiments directed to absorbent articles, such as diapers and hygiene products, garments, wound care, and the like, the basis weights of the nonwoven fabric may generally from about 5 grams per square meter (gsm) to about 200 gsm, and in particular, from about 15 to 50 gsm.

In some embodiments, for instance, the high loft nonwoven fabric may have a basis weight from about 8 gsm to about 70 gsm. In certain embodiments, for example, the fabric may comprise a basis weight from about 10 gsm to about 50 gsm. In further embodiments, for instance, the fabric may have a basis weight from about 11 gsm to about 30 gsm. As such, in certain embodiments, the fabric may have a basis weight from at least about any of the following: 7, 8, 9, 10, and 11 gsm and/or at most about 150, 100, 70, 60, 50, 40, and 30 gsm (e.g., about 9-60 gsm, about 11-40 gsm, etc.).

### A. Through Air Bonded High Loft Nonwoven

In certain embodiments, the high loft nonwoven fabric comprises a high loft spunbond nonwoven in which the continuous filaments are thermally bonded to each other via through air bonding, and in which the filaments have not been subjected to calender bonding, such as thermal point bonding, or other compaction processes that significantly reduce the loft of the high loft nonwoven fabric (e.g., a loss of greater than 10% in thickness of the nonwoven fabric).

The through air bonded high loft spunbond nonwoven typically exhibits an increase in loft (e.g., thickness) of at least 20% in comparison to a similar nonwoven fabric in which 1) the percent difference in basis weights between the high loft nonwoven fabric and the similar nonwoven fabric is less than 2%, and in particular, less than 1%, and more particularly, less than 0.5 percent, and 2) the percent difference in average fiber diameter between the high loft nonwoven and the similar nonwoven fabric is less than 5%, and in particular, less than 4%, and more particularly, less than 3 percent. The similar nonwoven fabric may have identical or substantially identical chemistry to the through air bonded high loft nonwoven fabric.

In certain embodiments, the high loft spunbond nonwoven is prepared using conventional spunbond techniques in which the filaments of the high loft spunbond nonwoven are bonded to adjacent filaments with through air bonding. In this way, the filaments are not subjected to bonding or compaction techniques which result in compaction of the nonwoven fabric, and hence, loss of loft of the fabric.

### B. High Loft Nonwoven Having Crimped Filaments

In certain embodiments, the high loft spunbond nonwoven comprises continuous crimped filaments. Generally, crimped continuous filaments comprise multicomponent filaments (e.g., bicomponent filaments) in which one of polymer components is different than the other polymer component (e.g., differences in crystallinity, molecular weight, melting temperature, polydispersity index, flexural modulus, heat of fusion, melt flow rate (MFR), and crimp inducing polymer additives, such as blends with meltblown resins and/or polymers having low isotacticity) so that the bicomponent filaments advantageously develop spontaneous or possess natural crimp. In certain embodiments, the crimped continuous filaments have a bicomponent configuration selected from the group consisting of side-by-side, eccentric sheath/core, D-centric sheath/core, or any other configuration capable of developing or possessing crimp.

In certain embodiments, the crimped continuous filaments have a helical crimp comprising a plurality of loops along the length of the filaments. Typically, the number of helical loops per cm may range from about 2 to 100, and in particular, from about 5 to 75. In a preferred embodiment, the crimped continuous filaments have at least 10 helical loops per cm, and more particularly, at least about 15 helical loops per cm. In a preferred embodiment, the number of crimps per cm is from about 2 to 20 helical loops.

A wide variety of different polymers may be used in the production of the crimped continuous filaments. In particular, the bicomponent fibers may comprise polyolefins, such as polypropylenes, polyethylenes, and combinations thereof. In addition, combinations of the polymers discussed in greater detail below (including both synthetic and bio-based polymers) may be used in the preparation of crimped continuous filaments. Additional polymer compositions that may be used to prepare crimped continuous filaments for use in certain embodiments of the invention are discussed in U.S. Patent Publication No. 2016/0221300, U.S. Pat. No. 6,454,989, European Patent No. 2 343 406 B1 and European Patent Application Nos. 3 121 314 and 3 246 443 the contents of all which are hereby incorporated by reference.

In certain embodiments, the crimped continuous filaments comprise a side-by-side configuration in which a first polymer component of the bicomponent fibers comprises a metallocene catalyzed polypropylene having an MFR from 19 to 40 g/min, and the second polymer component comprises a Ziegler-Natta catalyzed polypropylene having an MFR from about 20 to 35 g/10 min. Unless otherwise stated, MFR is measured in accordance with ISO 1133, 230° C./2.16 kg force.

### C. High Loft Nonwoven Having a High Loft Additive

In certain embodiments, the high loft nonwoven fabric exhibits an increased loft in which at least some of the fibers of the nonwoven fabric comprise a blend of a polymer resin and a high loft additive. In one embodiment, the present invention provides a nonwoven fabric comprising a plurality of fibers wherein the plurality fibers comprise a blend of a polymeric resin and at least one high loft additive. As explained in greater detail below, the inclusion of the high loft additive in the polymer resin improves the loft (e.g., thickness) of the fabric in comparison to an identical fabric that does not include the high loft additive.

In certain embodiments, the high loft additive comprises an aliphatic fatty acid amide having at least one amide group and at least one aliphatic carbon chain having from 10 to 22 carbon atoms. In particular, the high loft additive includes fatty acid amides of the general formula (1):

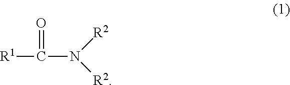

where R¹ comprises a C₁₀-C₂₂ carbon chain, which may be saturated or unsaturated and aliphatic, and R², independently, is selected from H, a C₁-C₄ alkyl group, R¹, and R³, with R³ having the following formula (2):

—(C₁-C₄)C(═O)N(R⁵)(R¹) (2)

wherein R⁵ is H or a C₁-C₄ alkyl group. In a preferred embodiment, R⁵ is H.

The a C₁₀-C₂₂ carbon chain of R¹ is generally linear although some chains may include some minor branching (e.g., C₁-C₄ side chain branching). Typically, the R¹ carbon chain will have from 10 to 22 carbon atoms, with a chain length of 14 to 20 carbon atoms being somewhat more preferred, and a chain length of 16 to 18 carbon atoms being most preferred.

In some embodiments, R¹ may include from 1 to 4 vinyl groups, 1 to 3 vinyl groups, 1 to 2 vinyl groups, and in particular, a single vinyl groups. In certain preferred embodiments, R¹ is saturated and does not include any vinyl groups.

The amide group of formula (1) may be primary, secondary, or tertiary. In a preferred embodiment, the amide group is a secondary amide.

In certain embodiments, the high loft additive comprises a fatty acid amide having two amide groups of the general formula (3)

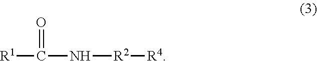

where R¹, independently, comprises an aliphatic carbon chain having from C₁₀-C₂₂, and R² is a C₁-C₄ group, and R⁴ is selected from —C(—O)NH(R¹), —C(═O)N(R¹)₂, and —C(═O)NH₂, —C(═O)N(R⁵)(R¹), and —C(═O)N(R⁵); where R⁵ is as defined previously.
The aliphatic C₁₀-C₂₂ carbon chain of R¹ is generally linear although some chains may include some minor branching (e.g., C₁-C₄ side chain branching). Typically, the C₁₀-C₂₂ chain will have from 10 to 18 carbon atoms, with an chain length of 14 to 20 carbon atoms being somewhat more preferred, and a chain length of 16 to 18 carbon atoms being most preferred. In the compound of formula (3), R¹ may be the same or different. As noted previously, R¹ may include 1 to 4 vinyl groups, and may include some minor branching (e.g., C₁-C₄ side chain branching). In a preferred embodiment, R¹ is saturated and does not include any vinyl groups. In addition, R¹ preferably does not include any branching.

In a preferred embodiment, the high loft additive comprises an aliphatic amide having two amide groups of the following formula (4):

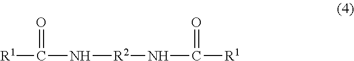

wherein each R¹, independently, comprises an aliphatic C₁₀-C₂₂ carbon chain, which may be saturated or unsaturated, and R² comprises a C₁-C₄ alkyl group. In certain embodiments of the compound of formula (4), the two R¹ groups are identical, and in other embodiments, the two R¹ groups are different from each other. In a preferred embodiment of formula (4), R¹ comprises a saturated aliphatic carbon chain having 14 to 18 carbon atoms. As discussed above, in certain embodiments R¹ may include 1 to 4 vinyl groups, and may include some minor branching (e.g., C₁-C₄ side chain branching). In a preferred embodiment, R¹ is saturated and does not include any vinyl groups. In addition, R¹ preferably does not include any branching.

In certain embodiments of the compound of formula (4), R² is selected from the group consisting of a methyl group, ethyl group, propyl group, and butyl group. In a preferred embodiment, R² is an ethyl group.

Examples of suitable fatty acid amides may include one or more of erucamide, oleamide, and stearamide behenamide, octadecane amide, ethylene bis-steramide, stearyl erucamide, and the like. In a preferred embodiment, the high loft additive comprises an aliphatic bis alkyl amide, such as N, N′-ethylene bis-stearamide. Commercial examples of such ethylene bis-stearamides may be obtained from CRODA Polymer Additives under the product name CRODAMIDE™ EBS and Sigma-Aldrich under the product number 434671.

Generally, it may be desirable for the high loft additive to have a melting temperature below the melting temperature of the polymer resin in which the high loft additive is blended and a decomposition temperature above the melting temperature of the polymer resin.

In certain embodiments, high loft additive may be provided in a masterbatch carrier resin. For example, in one embodiment, the high loft additive is provided in a polymer carrier resin that is blended with the polymer resin prior to spinning of the fibers.

Typically, the amount of high loft additive in the polymer resin masterbatch is from about 1 to 25 weight percent based on the total weight of the masterbatch, with an amount from 2 to 20 weight percent being somewhat more typical. The masterbatch may also include additional additives, such as one or more compatibilizers. A commercial example of a high loft additive that may be used in embodiments of the disclosure includes a EBS masterbatch available from Sandridge Color Corporation under the product number 22188, which is an ethylene bis-stearamide in a polypropylene masterbatch.

A wide variety of different polymer resins may be used to prepare nonwoven fabrics in accordance with embodiments of the invention. As discussed in greater detail below, the polymers may be so-called synthetic polymers, bio-polymers, or may include blends of polymers.

The amount of the high loft additive in the fibers will generally depend on where the high loft additive is present in the structure of the fibers, and the final desired properties of the nonwoven fabric. In general, the amount of the high loft additive may range from about 0.0125 weight percent to about 10 weight percent, based on the total weight of the polymeric component of the fiber in which the high loft additive is present. In one embodiment, the concentration of the fatty acid amide may range from about 0.1 to 6 weight percent, such as 0.2 to 3, weight percent, based on the total weight of the polymeric component of the fiber in which the high loft additive is present. In preferred embodiments, the concentration of the fatty acid amide may range from about 0.1 to 2 weight percent, more preferably from about 0.2 to 1.5 weight percent, and even more preferable 0.3 to 1.25 weight percent, with 0.8 to 1.2 weight percent, based on the total weight of the polymeric component of the fiber in which the high loft additive is present.

For example, in monocomponent fibers the weight percent of the high loft additive in the fibers will be based on the total weight of the fiber. In such a case, the amount high loft additive may range from about 0.0125 weight percent to about 2.5 weight percent, based on the total weight of the fiber. However, in the case of a bicomponent fiber, the weight percent of the high loft additive will be based on the total weight of the component in which the high loft additive is present. For example, in the case of a bicomponent fiber having a sheath to core weight ratio of 30:70, and in which the high loft additive is only present in the sheath, the weight percent of the high loft additive in the fiber may range from about 0.0125 weight percent to about 2.5 weight percent, based on the total weight of the sheath, which results in a weight percent of the high loft additive that is from 0.00375 to 0.750, based on the total weight of the fiber.

In one embodiment, the amount of the high loft additive may be at least about any one of the following: at least 0.0125, at least 0.0250, at least 0.0375, at least 0.050, at least 0.0625, at least 0.075, at least 0.100, at least 0.125, at least 0.150, at least 0.1875, at least 0.2, at least 0.2475, at least 0.25, at least 0.3 at least 0.375, at least 0.40, at least 0.495, at least 0.50, at least 0.60, at least 0.80, at least 0.9904, at least 1.0, at least 1.25, at least 1.2375, at least 1.5, at least 1.875, at least 2.0, at least 2.5, at least 3.0, at least 3.5, at least 4.0, at least 4.5, at least 5.0, at least 5.5, at least 6.0, at least 6.5, at least 7.0. at least 7.5. at least 8.0, at least 8.5. at least 9.0, at least 9.5, at least 9.6, at least 9.7, at least 9.8, and at least 9.9, based on the total weight of the polymeric component of the fiber in which the high loft additive is present.

In other embodiments, the amount of high loft additive may be less than about any one of the following: 0.0250, 0.0375, 0.050, 0.0625, 0.075, 0.100, 0.125, 0.150, 0.1875, 0.2, 0.2475. 0.25. 0.3. 0.375. 0.40, 0.495, 0.50, 0.60, 0.80, 0.9904, 1.0, 1.25, 1.2375, 1.5, 1.875, 2.0, 2.5, 3.0, 3.5, 4.0, 4.5, 5.0. 5.5. 6.0. 6.5, 7.0, 7.5, 8.0, 8.5, 9.0, 9.5 and 10 weight percent. It should also be recognized that the amount of the high loft additive present in a polymer component of the fiber also encompasses ranges between the aforementioned amounts.

In certain embodiments, the fibers have a bicomponent structure in which the core and sheath both comprise the same type of polymer, and the sheath includes the high loft additive that is present in an amount that is from about 0.1 to 1 weight percent, based on the total weight of the sheath component, and in particular, from about 0.1 to 0.75, and more particularly from about 0.2 to 0.6 weight percent, and even more particularly, from about 0.3 to 0.4 weight percent, based on the total weight of the sheath component. Although, the high loft additive has generally discussed as being present in a monocomponent fiber or the sheath of a bicomponent fiber, it should be recognized that other arrangements are within the embodiments of the present invention. For example, the high loft additive may be present in only the core and not the sheath of a bicomponent fiber, or the high loft additive may be present in both the sheath and the core.

As the amount of the High Loft Additive in the fibers may vary depending on the amount of the high loft additive in the masterbatch polymer, the structure of the fiber (e.g., monocomponent or bicomponent), and in the case of the bicomponent, the ratio of a first polymer component to a second component in the fiber, the following tables provide exemplary ranges of the high loft additive in various fiber structures and at various loadings of the high loft additive in the masterbatch polymer, and at various loadings of the masterbatch in the polymer carrier resin.

TABLE 1A
Amounts of the High Loft Additive (HLA) in the Sheath of a bicomponent fiber having
a sheath to core weight ratio of 50:50 at various HLA and Master Batch (MB) loadings

| | Amount of HLA in | Amount of HLA in | Amount of HLA in | Amount of HLA in |
|---|---|---|---|---|
| | Sheath at an | Sheath at an | Sheath at an | Sheath at an |
| Amount of | addition of 5% | addition of 10% | addition of 20% | addition of 25% |
| HLA in MB | MB to Sheath | MB to Sheath | MB to Sheath | MB to Sheath |
| (%) | polymer (%) | polymer (%) | polymer (%) | polymer (%) |
| 0.25% | 0.0125 | 0.025 | 0.050 | 0.0625 |
| 0.50% | 0.025 | 0.050 | 0.100 | 0.125 |
| 0.75% | 0.0375 | 0.075 | 0.150 | 0.1875 |
| 1.0% | 0.050 | 0.100 | 0.200 | 0.250 |
| 2.0% | 0.100 | 0.200 | 0.400 | 0.500 |
| 3.0% | 0.150 | 0.300 | 0.600 | 0.750 |
| 4.0% | 0.200 | 0.400 | 0.800 | 1.000 |
| 4.95% | 0.2475 | 0.495 | 0.9904 | 1.2375 |
| 5.0% | 0.250 | 0.500 | 1.00 | 1.2500 |
| 7.5% | 0.375 | 0.750 | 1.500 | 1.8750 |
| 10.0% | 0.500 | 1.000 | 2.000 | 2.5000 |

TABLE 1B
Amounts of the High Loft Additive (HLA) in the Fabric comprised of bicomponent fibers having
a sheath to core weight ratio of 50:50 at various HLA and Master Batch (MB) loadings

| | Amount of HLA in | Amount of HLA in | Amount of HLA in | Amount of HLA in |
|---|---|---|---|---|
| | Fabric at an | Fabric at an | Fabric at an | Fabric at an |
| Amount of | addition of 5% | addition of 10% | addition of 20% | addition of 25% |
| HLA in MB | MB to Sheath | MB to Sheath | MB to Sheath | MB to Sheath |
| (%) | polymer (%) | polymer (%) | polymer (%) | polymer (%) |
| 0.25% | 0.00625 | 0.0125 | 0.025 | 0.03125 |
| 0.50% | 0.01250 | 0.025 | 0.050 | 0.06250 |
| 0.75% | 0.01875 | 0.0375 | 0.075 | 0.09375 |
| 1.0% | 0.02500 | 0.050 | 0.100 | 0.12500 |
| 2.0% | 0.05000 | 0.100 | 0.200 | 0.25000 |
| 3.0% | 0.07500 | 0.150 | 0.300 | 0.37600 |
| 4.0% | 0.10000 | 0.200 | 0.400 | 0.50000 |
| 4.95% | 0.12375 | 0.2475 | 0.495 | 0.61875 |
| 5.0% | 0.12500 | 0.250 | 0.500 | 0.62500 |
| 7.5% | 0.18750 | 0.375 | 0.750 | 0.93750 |
| 10.0% | 0.25000 | 0.500 | 1.000 | 1.25000 |

TABLE 2A
Amounts of the High Loft Additive (HLA) in Sheath of a bicomponent fiber having a
sheath to core weight ratio of 30:70 at various HLA and Master Batch (MB) loadings

| | Amount of HLA in | Amount of HLA in | Amount of HLA in | Amount of HLA in |
|---|---|---|---|---|
| | Sheath at an | Sheath at an | Sheath at an | Sheath at an |
| Amount of | addition of 5% | addition of 10% | addition of 20% | addition of 25% |
| HLA in MB | MB to Sheath | MB to Sheath | MB to Sheath | MB to Sheath |
| (%) | polymer (%) | polymer (%) | polymer (%) | polymer (%) |
| 0.25% | 0.0125 | 0.025 | 0.050 | 0.0625 |
| 0.50% | 0.025 | 0.050 | 0.100 | 0.125 |
| 0.75% | 0.0375 | 0.075 | 0.150 | 0.1875 |
| 1.0% | 0.050 | 0.100 | 0.200 | 0.250 |
| 2.0% | 0.100 | 0.200 | 0.400 | 0.500 |
| 3.0% | 0.150 | 0.300 | 0.600 | 0.750 |
| 4.0% | 0.200 | 0.400 | 0.800 | 1.000 |
| 4.95% | 0.2475 | 0.495 | 0.9904 | 1.2375 |
| 5.0% | 0.250 | 0.500 | 1.00 | 1.2500 |
| 7.5% | 0.375 | 0.750 | 1.500 | 1.8750 |
| 10.0% | 0.500 | 1.000 | 2.000 | 2.5000 |

TABLE 2B
Amounts of the High Loft Additive (HLA) in a Fabric comprising bicomponent fibers having
a sheath to core weight ratio of 30:70 at various HLA and Master Batch (MB) loadings

| | Amount of HLA in | Amount of HLA in | Amount of HLA in | Amount of HLA in |
|---|---|---|---|---|
| | Fabric at an | Fabric at an | Fabric at an | Fabric at an |
| Amount of | addition of 5% | addition of 10% | addition of 20% | addition of 25% |
| HLA in MB | MB to Sheath | MB to Sheath | MB to Sheath | MB to Sheath |
| (%) | polymer (%) | polymer (%) | polymer (%) | polymer (%) |
| 0.25% | 0.00375 | 0.0075 | 0.015 | 0.01875 |
| 0.50% | 0.00750 | 0.0150 | 0.0300 | 0.0375 |
| 0.75% | 0.01125 | 0.0225 | 0.04500 | 0.05625 |
| 1.0% | 0.0150 | 0.0300 | 0.0600 | 0.0750 |
| 2.0% | 0.030 | 0.060 | 0.1200 | 0.1500 |
| 3.0% | 0.0450 | 0.0900 | 0.1800 | 0.2250 |
| 4.0% | 0.0600 | 0.1200 | 0.2400 | 0.3000 |
| 4.95% | 0.07425 | 0.1485 | 0.2970 | 0.37125 |
| 5.0% | 0.0750 | 0.1500 | 0.3000 | 0.375 |
| 7.5% | 0.1125 | 0.2250 | 0.4500 | 0.5625 |
| 10.0% | 0.1500 | 0.3000 | 0.6000 | 0.7500 |

TABLE 3
Amounts of the High Loft Additive (HLA)in a Fabric comprising monocomponent
fibers at various HLA and Master Batch (MB) loadings

| | Amount of HLA in | Amount of HLA in | Amount of HLA in | Amount of HLA in |
|---|---|---|---|---|
| Amount of | Fabric at an | Fabric at an | Fabric at an | Fabric at an |
| HLA in MB | addition of 5% | addition of 10% | addition of 20% | addition of 25% |
| (%) | MB (%) | MB (%) | MB (%) | (%) |
| 0.25% | 0.0125 | 0.025 | 0.050 | 0.0625 |
| 0.50% | 0.025 | 0.050 | 0.100 | 0.125 |
| 0.75% | 0.0375 | 0.075 | 0.150 | 0.1875 |
| 1.0% | 0.050 | 0.100 | 0.200 | 0.250 |
| 2.0% | 0.100 | 0.200 | 0.400 | 0.500 |
| 3.0% | 0.150 | 0.300 | 0.600 | 0.750 |
| 4.0% | 0.200 | 0.400 | 0.800 | 1.000 |
| 4.95% | 0.2475 | 0.495 | 0.9904 | 1.2375 |
| 5.0% | 0.250 | 0.500 | 1.600 | 1.25 |
| 7.5% | 0.375 | 0.750 | 1.500 | 1.875 |
| 10.0% | 0.500 | 1.000 | 2.000 | 2.500 |

In accordance with certain embodiments, the basis weight of the nonwoven fabric may vary depending on the end use application of the fabric. As noted previously, in embodiments directed to absorbent articles, such as diapers and hygiene products, garments, wound care, and the like, the basis weights of the nonwoven fabric may generally from about 5 grams per square meter (gsm) to about 200 gsm.

In some embodiments, for instance, the fabric may have a basis weight from about 8 gsm to about 100 gsm. In certain embodiments, for example, the fabric may comprise a basis weight from about 10 gsm to about 50 gsm. In further embodiments, for instance, the fabric may have a basis weight from about 11 gsm to about 30 gsm. As such, in certain embodiments, the fabric may have a basis weight from at least about any of the following: 7, 8, 9, 10, and 11 gsm and/or at most about 150, 100, 70, 60, 50, 40, and 30 gsm (e.g., about 9-60 gsm, about 11-40 gsm, etc.).

Advantageously, the addition of the high loft additive in the fibers provides significant increases in nonwoven fabric loft in comparison to an identical or similarly prepared nonwoven fabric that does not include the high loft additive. In this regard, nonwoven fabrics in accordance with embodiments of the present invention may exhibit an increase in thickness that are at least 25% greater in comparison to a similarly prepared nonwoven fabric that does not include the high loft additive. In some embodiments, the nonwoven fabric may exhibit an increase in thickness (caliper or loft) that is from 50% to 200% greater than the thickness of a similarly prepared nonwoven fabric that does not include the high loft additive. By “similarly prepared” it is meant that the nonwoven fabrics have identical chemistry or substantially identical chemistry and have been processed under similar or identical processing conditions, with the exception of the addition of the high loft additive in the inventive nonwoven fabrics.

In certain embodiments, nonwoven fabrics in accordance with embodiment the invention exhibit an increase in thickness (caliper) ranging from 10 to 1,000%, and in particular, from about 20 to 500%, and more particularly, from 100 to 250% in comparison to a similar nonwoven fabric in which the similar nonwoven comprises fibers that do not include the high loft additive.

In certain embodiments, the high loft nonwoven fabrics having the high loft additive may exhibit an increase in thickness that is from about 10 to 1000% in comparison to a similarly prepared nonwoven fabric that does not include the high loft additive. In a preferred embodiment, the high loft nonwoven fabric may exhibit an increase in thickness that is from about 80 to 500%, and more preferably, from about 140 to 480% in comparison to a similarly prepared nonwoven fabric that does not include the high loft additive. For example, the high loft nonwoven fabric may exhibit an increase in thickness of any one or more of at least 10%, at least 20%, at least 30%, at least 40%, at least 50%, at least 60%, at least 70%, at least, 80%, at least 90%, at least 100%, at least 125%, at least 150%, at least 175%, at least 200%, at least 225%, at least 250%, at least 275%, at least 300%, at least 325%, at least 350%, at least 375%, at least 400%, at least 425%, at least 450%, at least 475%, at least 500%, at least 525%, at least 550%, at least 575%, at least 600%, at least 625%, at least 650%, at 675%, at least 700%, at least 725%, at least 750%, at least 775%, at least 800%, at least 825%, at least 850%, at least 875%, at least 900%, at least 925%, at least 950, at least 975%, or at least 1,000%, in comparison to a similarly prepared nonwoven fabric that does not include the high loft additive.

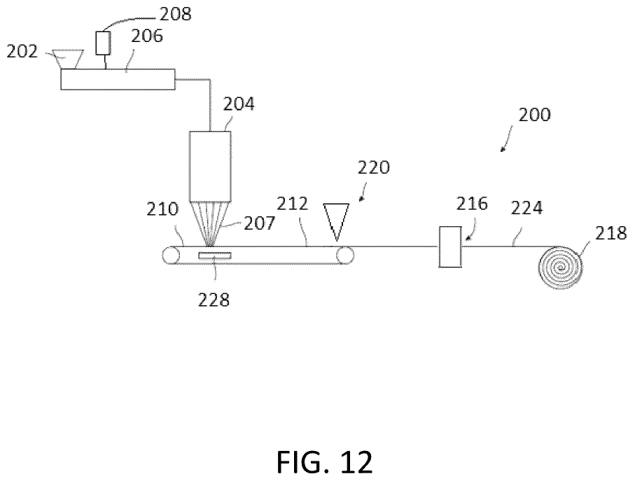

With reference to FIG. 12, for example, a schematic diagram of a high loft spundbond nonwoven preparation system in accordance with certain embodiments of the invention is illustrated and broadly designated by reference character 200. As shown in FIG. 12, a first polymer source (i.e. hopper) 202 is in fluid communication with a spunbond spin beam 204 via the extruder 206. It should be understood that other nonwoven forming systems may be used in accordance with certain embodiments of the invention, such as meltblown, carded, air bonded, resin bonded, spunlace, and the like.

In certain embodiments, an optional source of a high loft additive 208 is in fluid communication with either the hopper 202 or the extruder 206. The high loft additive may be preblended with the polymer or may be metered into the hopper or extruder.

In certain embodiments, the first polymer source may provide a stream of a molten or semi-molten polymer resin. Following extrusion, the extruded polymer stream containing the polymer (and optional) high loft additive are introduced into the spunbond spin beam 204 at which point a plurality of streams are introduced into a die head (not shown) of a spunbond spin beam. The die head includes a plurality of fluid orifices and one or more streams of air for drawing and attenuating the polymer streams as they exit the die head to produce a stream of spunbond fibers. The spin beam 204 produces a stream of continuous spunbond fibers 207 that are deposited on the collection surface 210 to produce a web of filaments. At this stage, the filaments may comprise a web 212 of filaments that are unbonded or slightly bonded to each other.

In certain embodiments, an optional bonding unit 216 is disposed downstream of the collection surface 210 and is configured and arranged to thermally bond fibers to each other to form a coherent web. During thermal bonding the web 212 of fibers, the fibers are heated to a temperature that is sufficient to soften at least one polymer component comprising fibers of web 212 to produce a bonded nonwoven fabric 224. In certain embodiments, the bonded high loft spunbond nonwoven 224 moves to a winder 218, where the nonwoven is then wound onto rolls.

In certain embodiments, the bonding unit comprises an air through bonder in which the fibers are exposed to one or more streams of heated gas, such as air.

In some embodiments, an optional hot air knife 220 stabilizes the web of fibers by subjecting the fibers to a stream of heated gas, such as air, prior to delivery to the winder 218 or the bonding unit 216 for bonding. The hot air knife that exposes the web 212 to a stream of heated gas to lightly bond and stabilize the web.

In some embodiments, the system 200 may further comprise a vacuum source 228 disposed below collection surface 210. Vacuum source 128 provides a vacuum that helps draw and pull the fibers 107 onto collection surface 110.

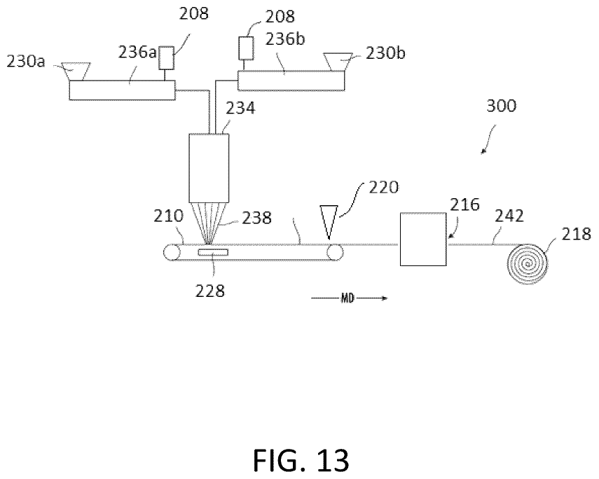

With reference to FIG. 13, a further aspect of a system and method of preparing a high loft spunbond nonwoven in accordance with at least one embodiment of the invention is illustrated and broadly designated by reference character 300. In this embodiment, system 300 may be configured and arranged to produce multicomponent meltblown fibers, such as bicomponent meltblown fibers.

System 300 includes a first polymer source (i.e. hopper) 230a that is in fluid communication with the spunbond spin beam 234 via the extruder 236a. A second polymer source (i.e. hopper) 230b is also in fluid communication with the spunbond spin beam 234 via extruder 236b. In the preparation of multicomponent fabrics, first polymer source may provide a stream of a first polymer resin, and the second polymer source may provide a stream of a second polymer resin. In melt spinning applications, the polymer streams are typically in a molten or semi-molten state. The first polymer resin and the second polymer resin may be different polymers, or may be the same polymers depending on the desired application and desired properties of the nonwoven fabric. For example, the first polymer resin may comprise a first polypropylene polymer and the second polymer resin may comprise a second polymer resin.

An optional source of a high loft additive 208 is in fluid communication with one of the extruders 236a, 236b, or with one of the hoppers 230a, 230b. The high loft additive may be preblended with the polymer or may be metered into the hopper or extruder. In FIG. 13, a source of high loft additive is shown in communication with both extruders 236a, 236b; however, it should be recognized that the high loft additive, when present, may be blended with one of the polymer resins in only one of the extruders, and not blended with a polymer resin in the second extruder.

Following extrusion, the extruded polymer streams are introduced into the spunbond spin beam 234 at which point the plurality of polymer streams are introduced into a die head (not shown) of a meltblown spin beam. The die head includes a plurality of fluid orifices and one or more streams of gas, such as air, for drawing and attenuating the polymer streams as they exit the die head to produce a stream of meltblown fibers.

The spin beam 234 produces a plurality of multicomponent meltblown fibers 238 that are deposited on the collection surface 210 to produce a web 240 of spunblowm fibers. At this stage, the meltblown web may comprise a web 140 of multicomponents fibers that are unbonded or slightly bonded to each other.

In certain embodiments, an optional bonding unit 216 is disposed downstream of the collection surface 210 and is configured and arranged to thermally bond filaments to each other to form a coherent web. During thermal bonding the web 240 of fibers, the fibers are heated to a temperature that is sufficient to soften at least one polymer component comprising fibers of web 240 to produce a bonded high loft spunbond nonwoven 224. In certain embodiments, the bonded or non-bonded nonwoven fabric moves to a winder 218, where the nonwoven is then wound onto rolls.

In certain embodiments, the bonding unit comprises an air through bonder in which the fibers are exposed to one or more streams of heated gas, such as air.

As in the previously discussed embodiment, system 300 may also include an optional hot air knife 220, and vacuum source 228.

### Carded Nonwoven Fabric Layer

In certain embodiments, the nonwoven substrate layer comprises a carded nonwoven fabric comprising a plurality of staple fibers that are substantially oriented in the same direction, such as in the machine direction. The plurality of staple fibers are bonded together to form a coherent web. In certain embodiments, the cared nonwoven fabric is through air bonded or resin bonded.

The inventors have discovered that the use of carded nonwoven fabrics having basis weights about 16 gsm or greater helps to improve the fluid management properties of the composite sheet material, and hence, the fluid management properties of the absorbent article comprising the composite sheet material.

In particular, it has been discovered that carded nonwoven fabrics having basis weights greater than about 16 gsm are generally characterized by improvements in resiliency, porosity, and thickness or loft. As a result, the carded fabrics provide improvements in fluid management properties include faster fluid in-take and transportation of fluids away from the skin of the wearer.

Preferably, the staple fibers of the carded nonwoven are substantially oriented/aligned in the same direction, such as in the machine direction of the carded nonwoven. Advantageously, a carded nonwoven has found to provide improved fluid management properties. In particular, a composite sheet material in accordance with certain embodiments of the invention exhibit improvements in fluid acquisition time and fluid rewet following multiple fluid insults.

The basis weight of the carded fabric layer may range from about 16 to 100 gsm, and in particular, from about 20 to 80 gsm, and more particularly, from about 25 to 60 gsm. In a preferred embodiment, the carded fabric layer has a basis weight that is about 25 to 55 gsm.

Generally, staple fibers that may be used in embodiments of the invention may have lengths ranging from about 10 to 65 mm, and in particular, from about 20 to 50 mm, and more particularly, from about 30 to 45 mm. In a preferred embodiment, the staple fibers have a length from about 35 to 51 mm.

In a preferred embodiment, the nonwoven substrate layer comprises a carded nonwoven fabric comprising a through air bonded fabric or a resin bonded fabric. Advantageously, it has been discovered that the use of through air bonded or resin bonded carded fabrics also helps to improve the fluid management properties of the resulting composite sheet material. During through air bonding and/or resin bonding of the staple fibers, the carded nonwoven fabric is not compacted or otherwise compressed. As a result, the carded fabric retains most, if not all, of its resiliency, porosity, density, and loft (e.g., thickness). In contrast, other bonding techniques, such as thermal point bonding or thermal calender bonding, may result in some compression and compaction of the carded fabric. This in turn, may decrease the desirable properties (e.g., resiliency, loft, porosity, and density) of the carded nonwoven fabric, and hence, negatively affect the fluid management properties of the carded nonwoven fabric. In certain embodiments, the carded nonwoven fabric is characterized by the absence of thermal point bonding or thermal calender bonding.

It should be recognized that in some embodiments of the invention, it may be desirable to thermally bond the carded nonwoven fabric using thermal point bonding or thermal calender bonding techniques, although not necessarily with equivalent results.

Staple fibers that may be used in embodiments of the invention may be monocomponent or multicomponent. The staple fibers may also comprise natural fibers including cellulose-based fibers, synthetic fibers, fibers derived from bio-based polymers, and combinations thereof.

In one embodiment, the staple fibers comprise multicomponent fibers having at least two polymer components arranged in structured domains across the cross section of the fiber. As is generally well known to those skilled in the art, polymer domains or components are arranged in substantially continuously positioned zones across the cross-section of the multicomponent fiber and extend continuously along the length of the multicomponent fiber. More than two components could be present in the multicomponent fiber. A preferred configuration is a sheath/core arrangement wherein a first component, the sheath, substantially surrounds a second component, the core. The resulting sheath/core bicomponent fiber may have a round or non-round cross-section. Other structured fiber configurations as known in the art can be used including side-by-side, segmented pie, islands-in-the-sea and tipped multilobal structures.

In certain embodiments, the fibers are bicomponent in which a first polymer component defines a sheath of the fiber, and a second polymer component defines a core of the fiber. Generally, the weight percentage of the sheath to that of the core in the fibers may vary widely depending upon the desired properties of the carded nonwoven fabric. For example the weight ratio of the sheath to the core may vary between about 5:95 to 95:5, such as from about 10:90 to 90:10, and in particular from about 20:80 to 80:20. In a preferred embodiment, the weight ratio of the sheath to the core is about 25:75 to 35:65, with a weight ratio of about 20:80 to 50:50 being preferred.

A wide variety of polymers may be used for preparing staple fibers for use in the carded nonwoven layer and for preparing continuous filaments for use in the high loft spunbond nonwoven fabric. Examples of suitable fibers include may include polyolefins, such as polypropylene and polyethylene, and copolymers thereof, polyesters, such as polyethylene terephthalate (PET), polytrimethylene terephthalate (PTT), and polybutylene terephthalate (PBT), nylons, polystyrenes, copolymers, and blends thereof, and other synthetic polymers that may be used in the preparation of fibers.

In one embodiment, the fibers/filaments have a sheath/core configuration comprising a polyethylene sheath and a polypropylene core. In other embodiments, the fibers/filaments may have a sheath/core configuration comprising a polyethylene sheath and a polyester core, such as a core comprising polyethylene terephthalate.

The above noted polymers are generally considered to be derived from synthetic sources, such as a petroleum derived polymer. In some embodiments, it may be desirable to provide a nonwoven substrate layer comprising one or more sustainable polymer components. In contrast to polymers derived from petroleum sources, sustainable polymers are generally derived from a bio-based material. In some embodiments, a sustainable polymer may also be considered biodegradeable. A special class of biodegradable product made with a bio-based material might be considered as compostable if it can be degraded in a composing environment. The European standard EN 13432. “Proof of Compostability of Plastic Products” may be used to determine if a fabric or film comprised of sustainable content could be classified as compostable.

In one such embodiment, the fibers of the nonwoven substrate layer comprise a bio-based polymer. In certain embodiments, the fibers are substantially free of synthetic materials, such as petroleum-based materials and polymers. For example, fibers comprising the carded nonwoven layer may have less than 25 weight percent of materials that are non-bio-based, and more preferably, less than 20 weight percent, less than 15 weight percent, less than 10 weight percent, and even more preferably, less than 5 weight percent of non-bio-based materials, based on the total weight of the nonwoven substrate layer.

In one embodiment, bio-based polymers for use may include aliphatic polyester based polymers, such as polylactic acid, and bio-based derived polyethylene.

Aliphatic polyesters useful in the present invention may include homo-and copolymers of poly (hydroxyalkanoates), and homo-and copolymers of those aliphatic polyesters derived from the reaction product of one or more polyols with one or more polycarboxylic acids that are typically formed from the reaction product of one or more alkanediols with one or more alkanedicarboxylic acids (or acyl derivatives). Polyesters may further be derived from multifunctional polyols, e.g. glycerin, sorbitol, pentaerythritol, and combinations thereof, to form branched, star, and graft homo-and copolymers. Polyhydroxyalkanoates generally are formed from hydroxyacid monomeric units or derivatives thereof. These include, for example, polylactic acid, polyhydroxybutyrate, polyhydroxyvalerate, polycaprolactone and the like. Miscible and immiscible blends of aliphatic polyesters with one or more additional semicrystalline or amorphous polymers may also be used.

One useful class of aliphatic polyesters are poly (hydroxyalkanoates), derived by condensation or ring-opening polymerization of hydroxy acids, or derivatives thereof. Suitable poly (hydroxyalkanoates) may be represented by the formula: H(O—R—C(O)—)ₙOH where R is an alkylene moicty that may be linear or branched having 1 to 20 carbon atoms, preferably 1 to 12 carbon atoms optionally substituted by catenary (bonded to carbon atoms in a carbon chain) oxygen atoms; n is a number such that the ester is polymeric, and is preferably a number such that the molecular weight of the aliphatic polyester is at least 10,000, preferably at least 30,000, and most preferably at least 50,000 daltons. In certain embodiments, the molecular weight of the aliphatic polyester is typically less than 1,000,000, preferably less than 500,000, and most preferably less than 300,000 daltons. R may further comprise one or more caternary (i.e. in chain) ether oxygen atoms. Generally, the R group of the hydroxy acid is such that the pendant hydroxyl group is a primary or secondary hydroxyl group.

Useful poly(hydroxyalkanoates) include, for example, homo-and copolymers of poly(3-hydroxybutyrate), poly(4-hydroxybutyrate), poly(3-hydroxyvalerate), poly(lactic acid) (as known as polylactide), poly(3-hydroxypropanoate), poly(4-hydropentanoate), poly(3-hydroxypentanoate), poly(3-hydroxyhexanoate), poly(3-hydroxyheptanoate), poly(3-hydroxyoctanoate), polydioxanone, polycaprolactone, and polyglycolic acid (i.e. polyglycolide). Copolymers of two or more of the above hydroxy acids may also be used, for example, poly(3-hydroxybutyrate-co-3-hydroxyvalerate), poly(lactate-co-3-hydroxypropanoate), poly(glycolide-co-p-dioxanone), and poly(lactic acid-co-lycolic acid). Blends of two or more of the poly(hydroxyalkanoates) may also be used, as well as blends with one or more semicrystalline or amorphous polymers and/or copolymers.

The aliphatic polyester may be a block copolymer of poly(lactic acid-co-glycolic acid). Aliphatic polyesters useful in the inventive compositions may include homopolymers, random copolymers, block copolymers, star-branched random copolymers, star-branched block copolymers, dendritic copolymers, hyperbranched copolymers, graft copolymers, and combinations thereof.

Another useful class of aliphatic polyesters includes those aliphatic polyesters derived from the reaction product of one or more alkanediols with one or more alkanedicarboxylic acids (or acyl derivatives). Such polyesters have the general formula:

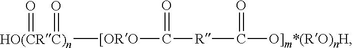

where R′ and R″ each represent an alkylene moiety that may be linear or branched having from 1 to 20 carbon atoms, preferably 1 to 12 carbon atoms, and m is a number such that the ester is polymeric, and is preferably a number such that the molecular weight of the aliphatic polyester is at least 10,000, preferably at least 30,000, and most preferably at least 50,000 daltons, but less than 1,000,000, preferably less than 500,000 and most preferably less than 300,000 daltons. Each n is independently 0 or 1. R′and R″ may further comprise one or more caternary (i.e. in chain) ether oxygen atoms.

Examples of aliphatic polyesters include those homo-and copolymers derived from (a) one or more of the following diacids (or derivative thereof): succinic acid; adipic acid; 1,12 dicarboxydodecane; fumaric acid; glutartic acid; diglycolic acid; and maleic acid; and (b) one of more of the following diols: ethylene glycol; polyethylene glycol; 1,2-propane diol; 1,3-propanediol; 1,2-propanediol; 1,2-butanediol; 1,3-butanediol; 1,4-butanediol; 2,3-butanediol; 1,6-hexanediol; 1,2 alkane diols having 5 to 12 carbon atoms; diethylene glycol; polyethylene glycols having a molecular weight of 300 to 10,000 daltons, and preferably 400 to 8,000 daltons; propylene glycols having a molecular weight of 300 to 4000 daltons; block or random copolymers derived from ethylene oxide, propylene oxide, or butylene oxide; dipropylene glycol; and polypropylene glycol, and (c) optionally a small amount, i.e., 0.5-7.0 mole percent of a polyol with a functionality greater than two, such as glycerol, neopentyl glycol, and pentaerythritol.

Such polymers may include polybutylene succinate homopolymer, polybutylene adipate homopolymer, polybutyleneadipate-succinate copolymer, polyethylenesuccinate-adipate copolymer, polyethylene glycol succinate homopolymer and polyethylene adipate homopolymer.

Commercially available aliphatic polyesters include poly(lactide), poly(glycolide), poly(lactide-co-glycolide), poly(L-lactide-co-trimethylene carbonate), poly(dioxanone), poly(butylene succinate), and poly(butylene adipate).

The term “aliphatic polyester” covers—besides polyesters which are made from aliphatic and/or cycloaliphatic components exclusively also polyesters which contain besides aliphatic and/or cycloaliphatic units, aromatic units, as long as the polyester has substantial sustainable content.

In addition to PLA based resins, the nonwoven substrate layer may include other polymers derived from an aliphatic component possessing one carboxylic acid group and one hydroxyl group, which are alternatively called polyhydroxyalkanoates (PHA). Examples thereof are polyhydroxybutyrate (PHB), poly-(hydroxybutyrate-co-hydroxyvaleterate) (PHBV), poly-(hydroxybutyrate-co-polyhydroxyhexanoate) (PHBH), polyglycolic acid (PGA), poly-(epsilon-caprolactione) (PCL) and preferably polylactic acid (PLA).

Examples of additional polymers that may be used in embodiments of the invention include polymers derived from a combination of an aliphatic component possessing two carboxylic acid groups with an aliphatic component possessing two hydroxyl groups, and are polyesters derived from aliphatic diols and from aliphatic dicarboxylic acids, such as polybutylene succinate (PBSU), polyethylene succinate (PESU), polybutylene adipate (PBA), polyethylene adipate (PEA), polytetramethy-lene adipate/terephthalate (PTMAT).

Useful aliphatic polyesters include those derived from semicrystalline polylactic acid. Poly(lactic acid) or polylactide (PLA) has lactic acid as its principle degradation product, which is commonly found in nature, is non-toxic and is widely used in the food, pharmaceutical and medical industries. The polymer may be prepared by ring-opening polymerization of the lactic acid dimer, lactide. Lactic acid is optically active and the dimer appears in four different forms: L,L-lactide, D,D-lactide, D,L-lactide (meso lactide) and a racemic mixture of L,L- and D,D-. By polymerizing these lactides as pure compounds or as blends, poly(lactide) polymers may be obtained having different stereochemistries and different physical properties, including crystallinity. The L,L- or D,D-lactide yields semicrystalline poly(lactide), while the poly(lactide) derived from the D,L-lactide is amorphous.

Generally, polylactic acid based polymers are prepared from dextrose, a source of sugar, derived from field corn. In North America corn is used since it is the most economical source of plant starch for ultimate conversion to sugar. However, it should be recognized that dextrose can be derived from sources other than corn. Sugar is converted to lactic acid or a lactic acid derivative via fermentation through the use of microorganisms. Lactic acid may then be polymerized to form PLA. In addition to corn, other agricultural based sugar sources may be used including rice, sugar beets, sugar cane, wheat, cellulosic materials, such as xylose recovered from wood pulping, and the like.

The polylactide preferably has a high enantiomeric ratio to maximize the intrinsic crystallinity of the polymer. The degree of crystallinity of a poly(lactic acid) is based on the regularity of the polymer backbone and the ability to crystallize with other polymer chains. If relatively small amounts of one enantiomer (such as D-) is copolymerized with the opposite enantiomer (such as L-) the polymer chain becomes irregularly shaped, and becomes less crystalline. For these reasons, when crystallinity is favored, it is desirable to have a poly(lactic acid) that is at least 85% of one isomer, at least 90% of one isomer, or at least 95% of one isomer in order to maximize the crystallinity.

In some embodiments, an approximately equimolar blend of D-polylactide and L-polylactide is also useful. This blend forms a unique crystal structure having a higher melting point (about 210° C.) than does either the D-poly(lactide) and L-(polylactide) alone (about. 190° C.), and has improved thermal stability.

Copolymers, including block and random copolymers, of poly(lactic acid) with other aliphatic polyesters may also be used. Useful co-monomers include glycolide, beta-propiolactone, tetramethylglycolide, beta-butyrolactone, gamma-butyrolactone, pivalolactone, 2-hydroxybutyric acid, alpha-hydroxyisobutyric acid, alpha-hydroxyvaleric acid, alpha-hydroxyisovaleric acid, alpha-hydroxycaproic acid, alpha-hydroxyethylbutyric acid, alpha-hydroxyisocaproic acid, alpha-hydroxy-beta-methylvaleric acid, alpha-hydroxyoctanoic acid, alpha-hydroxydecanoic acid, alpha-hydroxymyristic acid, and alpha-hydroxystearic acid.

Blends of poly(lactic acid) and one or more other aliphatic polyesters, or one or more other polymers may also be used. Examples of useful blends include poly(lactic acid) and poly(vinyl alcohol), polyethylene glycol/polysuccinate, polyethylene oxide, polycaprolactone and polyglycolide.

In certain preferred embodiments, the aliphatic polyester component comprises a PLA based resin. A wide variety of different PLA resins may be used to prepare nonwoven fabrics in accordance with embodiments of the invention. The PLA resin should have proper molecular properties to be spun in spunbond processes. Examples of suitable include PLA resins are supplied from Nature Works LLC, of Minnetonka, Minn. 55345 such as, grade 6752D, 6100D, and 6202D, which are believed to be produced as generally following the teaching of U.S. Pat. Nos. 5,525,706 and 6,807,973 both to Gruber et al. Other examples of suitable PLA resins may include L130, L175, and LX175, all from Corbion of Arkelsedijk 46, 4206 A C Gorinchem, the Netherlands.

In some embodiments, the nonwoven substrate layer may comprise sustainable polymer components of biodegradable products that are derived from an aliphatic component possessing one carboxylic acid group (or a polyester forming derivative thereof, such as an ester group) and one hydroxyl group (or a polyester forming derivative thereof, such as an ether group) or may be derived from a combination of an aliphatic component possessing two carboxylic acid groups (or a polyester forming derivative thereof, such as an ester group) with an aliphatic component possessing two hydroxyl groups (or a polyester forming derivative thereof, such as an ether group).

Additional nonlimiting examples of bio-based polymers include polymers directly produced from organisms, such as polyhydroxyalkanoates (e.g., poly(beta-hydroxyalkanoate), poly(3-hydroxybutyrate-co-3-hydroxyvalerate, NODAX™), and bacterial cellulose; polymers extracted from plants and biomass, such as polysaccharides and derivatives thereof (e.g., gums, cellulose, cellulose esters, chitin, chitosan, starch, chemically modified starch), proteins (e.g., zein, whey, gluten, collagen), lipids, lignins, and natural rubber; and current polymers derived from naturally sourced monomers and derivatives, such as bio-polyethylene, bio-polypropylene, polytrimethylene terephthalate, polylactic acid, NYLON 11, alkyd resins, succinic acid-based polyesters, and bio-polyethylene terephthalate.

In some embodiments, the bio-based polymer may comprise bio-based polyethylene that is derived from a biological source. For example, bio-based polyethylene can be prepared from sugars that are fermented to produce ethanol, which in turn is dehydrated to provide ethylene. An example of a suitable sugar cane derived polyethylene is available from Braskem S.A. under the product name PE SHA7260.

In some embodiments, the staple fibers for use in the carded nonwoven layer may comprise natural staple fibers. Generally, natural fibers are derived from plants or animals. Natural fibers derived from plants typically comprise cellulose materials, and may include cotton fibers, bamboo fibers, flax fibers, hemp fibers, grass fibers, such as elephant grass, jute fibers, abaca fibers, coir fibers, ramie fibers (also known as Chinese grass), sisal fibers, and the like.

Natural fibers derived from animals may include wool, silk, camel hair, alpaca wool, cashmere, angora wool, and the like. In a preferred embodiment, the natural fibers comprise cotton fibers.

A wide variety of different cellulose materials may be used for providing cellulose staple fibers. Fibers from Esparto grass, bagasse, kemp, flax, and other lignaceous and cellulose fiber sources may be utilized. Other fibers include absorbent natural fibers made from regenerated cellulose, polysaccharides or other absorbent fiber-forming compositions. In certain embodiments, the staple fibers may comprise bleached or non-bleached cotton fibers having fiber lengths ranging from about 15 to 38 mm. Examples of cotton fibers for use to form such carded fabrics include fibers sold under the product name TRUECOTTON® available from TJ Beall Company. It is noted that the non-bleached cotton fibers are easier to process in the carding process in comparison to bleached cotton fibers. However, in some embodiments bleached cotton fibers may be used, but not necessarily with equivalent results.

### Optional Components

In some embodiments, it may also be useful to optionally treat the nonwoven substrate layer with finishes containing functional additives or other chemicals, such as antimicrobial agents, flame retardant agents, catalysts, lubricants, softeners, surfactants including hydrophilic and hydrophobic compositions, light stabilizers, antioxidants, colorants such as dyes and/or pigments, antistatic agents, fillers, odor control agents, perfumes and fragrances, and the like, and combinations thereof. Other optional components may be included in the compositions described herein.

### Bio-Based Composite Sheet Material

In further aspects of the invention, bio-based composite sheet materials having improved fluid management properties are provided. In particular, embodiments of the present invention arc directed to a composite sheet material having a high bio-based material content. Preferably, bio-based composite sheet material in accordance with certain embodiments of the present invention have a bio-based material content of at least 90 weight % of the absorbent article, such as comprising a bio-based material content that is at least 91%, 92%, 93%, 94%, 95%, 96%, 97%, 98%, 99% or 100% by weight of the nonwoven fabric.

In some embodiments, the bio-based composite sheet material may comprise bio-based or biodegradable polymer materials. “Biodegradable” refers to a material or product which degrades or decomposes under environmental conditions that include the action of microrganisms. Thus a material is considered as biodegradable if a specified reduction of tensile strength and/or of peak elongation of the material or other critical physical or mechanical property is observed after exposure to a defined biological environment for a defined time. Depending on the defined biological conditions, a product comprised of a bio-based material might or might not be considered biodegradable.

Preferably, bio-based composite sheet materials in accordance with embodiments of the present invention are substantially free of synthetic materials, such as petroleum-based materials and polymers. For example, bio-based nonwoven fabrics in accordance with certain embodiments of the present invention have less than 10 weight percent of materials that are non-bio-based, and more preferably, less than 5 weight percent, less than 4 weight percent, less than 3 weight percent, and even more preferably, less than 1 weight percent of non-bio-based materials. based on the total weight of the bio-based nonwoven fabric

Preferably, the bio-based composite sheet material comprises bio-based polymers that are sourced from a biological source. Examples of bio-based polymers that may be used in certain aspects of the invention include the aliphatic polyesters and bio-based polymers previously described above.

In one embodiment, the film layer comprises a bio-based polymer, such as sugar can derived polyethylene, and the carded nonwoven layer comprises staple fibers comprising a bio-based polymer, natural fibers, and combinations thereof.

### Fabric Properties

Advantageously, nonwoven fabrics in accordance with certain embodiments of the invention exhibit improved fluid management properties. In the context of the invention, fluid management refers generally to the fabric's ability to quickly transport fluid through the thickness of the composite sheet material in one direction while at the same time preventing or retarding the flow of fluid back through the material in the opposite direction. In determining a fabric's fluid management properties, three tests are often utilized; fluid strikethrough, rewet, and run-off.

Fluid acquisition time measures the amount of time at which an insult of fluid on the surface of the fabric is absorbed into the absorbent article. Typically, fluid acquisition times are measured in a series of successive fluid insults on the surface of the fabric. Shorter fluid acquisition time through the absorbent article are indicative of the articles's ability to quickly move fluid away from the skin of the wearer and into an absorbent core.

Rewet measures the capacity of a nonwoven fabric to hold back fluids under pressure. Low rewet values are indicative of the fabric's ability to prevent a fluid from being transported back through the fabric where it could contact the skin of the wearer.

Certain embodiments of the invention exhibit a good balance in fluid management properties as evidenced by strikethrough, rewet, and run-off values. In certain embodiments, composite sheet materials in accordance with embodiments of the invention exhibit average fluid acquisition times of less than 5 seconds after a first fluid insult, less than 7 seconds after a second fluid insult. In additional embodiments, composite sheet materials in accordance with embodiments of the invention exhibit average fluid acquisition times of less than 3 seconds after a first fluid insult, less than 5 seconds after a second fluid insult. Repeated strike through acquisition times can be determined in accordance with NWSP 0.70.7.R0 (15).

In some embodiments, the composite sheet material may exhibit an average fluid acquisition time of less than 1 seconds, and in particular, less than any one or more of less than 0.8 seconds, less than 0.7 seconds, less than 0.6 seconds, less than 0.5 seconds, less than 0.4 seconds, less than 0.3 seconds, and less than 0.25 seconds. In certain embodiments, the composite sheet material may exhibit an single average fluid acquisition time from about 0.2 to 3 seconds, and in particular, from about 0.2 to 1 seconds. Single strike through acquisition times can be determined in accordance with NWSP 0.70.3.R0 (15).

In certain embodiments, composite sheet materials in accordance with the invention may exhibit a fluid rewet value of less than 1.0 gram (g), less than 0.5 g, less than 0.1 g, and in particular less than 0.08 g, and more particularly, less than 0.07 g. In certain embodiments, the composite sheet material exhibits a fluid rewet value ranging from about 0.05 to 1.0 g, and in particular, from about 0.06 to 0.08 g. Rewet values can be determined in accordance with NWSP 0.80.1.R0 (15) (single strike through) and NWSP 0.70.8.R0 (15) (repeated strike through).

In one particular embodiment, a composite sheet material according to an embodiment of the invention exhibits a fluid acquisition time after a first fluid insult of less than 1 seconds and a rewet of less than 0.15 grams, and in particular, a fluid acquisition time after a first fluid insult of less than 0.5 seconds and a rewet of less than 0.08 grams.

### System and Method for Preparing the Nonwoven Fabric

Certain embodiments according to the invention provide systems and methods for preparing a composite sheet material.

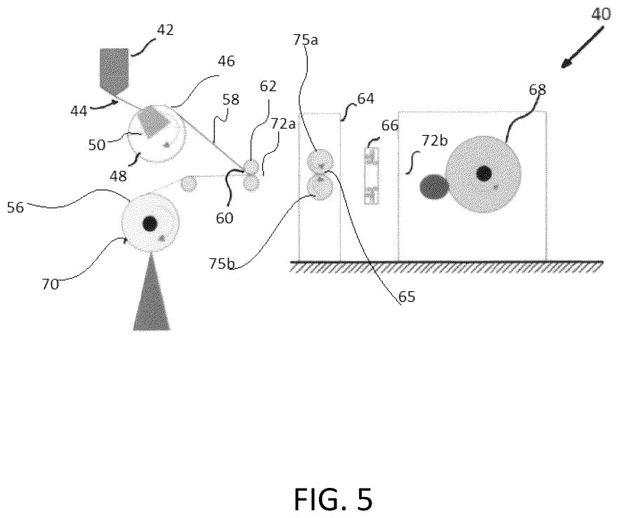

With reference to FIG. 5, for example, a schematic diagram of an apparatus for preparing the composite sheet material in accordance with certain embodiments of the invention is illustrated and broadly designated by reference character 40.

The apparatus 40 includes a film extrusion die 42 that extrudes a polymer melt curtain 44 onto a forming structure 46 that rotates about a cylinder 48 having a vacuum slot 50 through which a vacuum is pulled. As the polymer melt curtain passes over the vacuum slot, the forming structure on the cylinder forms apertured protuberances comprising a plurality of raised projections extending outwardly from the film. Typically, the vacuum extends laterally across a length of the cylinder and is in fluid communication with a vacuum source.

The now formed film layer 58 while still in a molten or semi-molten state is brought into contact with the nonwoven web 56 at point 60. In the illustrated embodiment, a nonwoven web 56 is unwound from a supply roll 70.

In the embodiment illustrated in FIG. 5, the apertured protuberances formed in the film extend from the surface of the film in the direction of the carded nonwoven web 56 (see, for example, the composite sheet material of FIG. 3),

Preferably, the formed film layer 58 having the apertured protuberances is still in a molten or semi-molten state when brought into contact with the nonwoven web 56 so that the fibers of the nonwoven web adjacent to the formed film layer 58 embed into the surface of the film as the two layers pass between the pair of rollers 62, 60.

The resulting composite sheet 72a travels to one or more subsequent rollers until it may be wound onto a roll 68. Additional rollers and/or other pieces of equipment may be used in the apparatus 40.

In some embodiments, the apparatus 40 may include an optional bonding unit 64 to facilitate bonding of the formed film layer and the nonwoven web. In one embodiment, the bonding unit may comprise a calender bonding unit 65 comprising a pair of cooperating rolls 75a, 75b in which one of the rolls is heated to a temperature sufficient to bond the formed film layer to the fibers of the carded nonwoven web 56. In some embodiments, one of the rolls may be an anvil roll while the other roll has raised projections to facilitate point bonding of fabric, and to impart a desired pattern to the composite sheet material. Examples of suitable bonding patterns may include oval, cross-direction rod (CD rod), honeycomb, diamond, square, rectangular, circle, or the like.

When subjected to thermal bonding, the composite sheet material is typically heated to a temperature sufficient to cause softening or the polymer comprising the formed film layer. In embodiments in which the formed film layer comprises polyethylene, the composite sheet material is typically heated to a temperature from about 110 to 130° C.

The illustrated embodiment is not intended to be limiting in any way. For example, in an embodiment, the apparatus 40 may also include additional equipment, such as intermeshing gears that may be used to activate the fluid distribution material in the machine direction or the transverse direction, if desired. Other equipment that may be included in the apparatus 40 include, but are not limited to, corona treatment apparatus, printers, festooning equipment, spooling equipment, and additional processing equipment that may emboss or provide additional apertures to the composite sheet material.

In some embodiments, the apparatus 40 may optionally include a cooling unit 66 configured to subject the bonded composite sheet material 72b to cooled gas to cool the polymeric material. In one embodiment, the cooled gas may comprise air that is at or near room temperature. In other embodiments, the cooling unit 66 may comprise one or more chilling rolls which are maintained at a reduced temperature to rapidly cool the formed film layer.

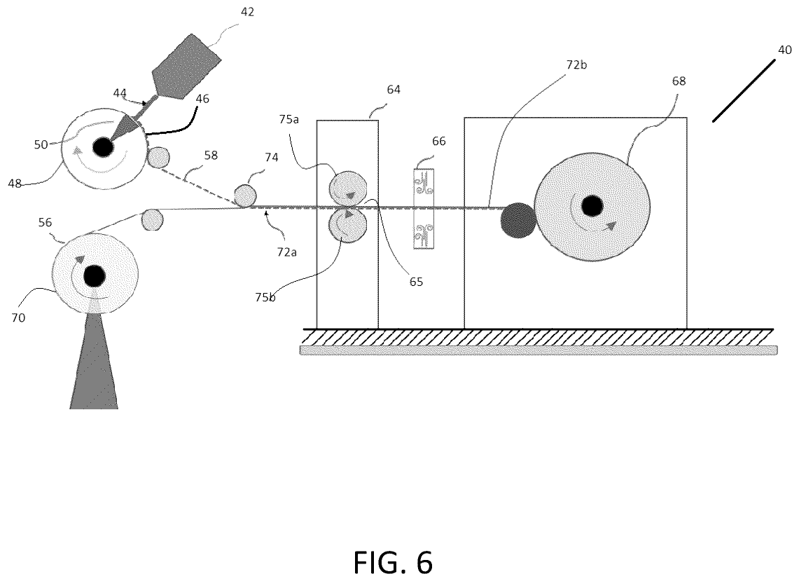

With reference to FIG. 6, further embodiment of the apparatus 40 for preparing the composite sheet material is shown. The apparatus of FIG. 6 is similar to that of FIG. 5, with the exception that the system includes roll 74 which is configured to pull the formed film layer 58 and nonwoven web 56 together.

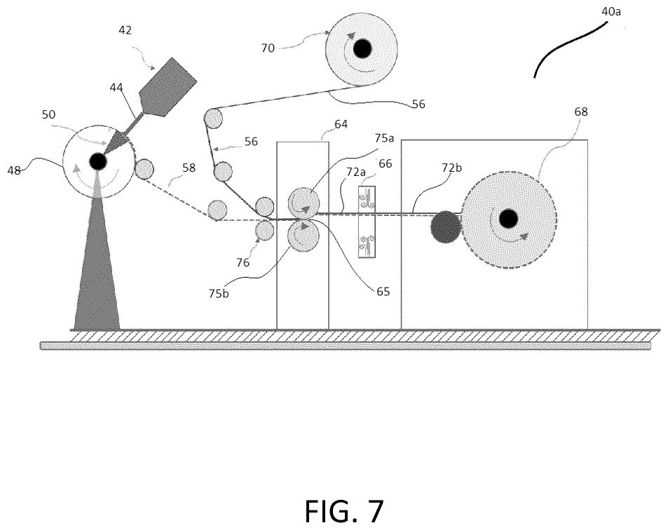

FIG. 7 illustrates a further embodiment of an apparatus 40a for preparing a composite sheet material in accordance with at least one embodiment of the invention. In this embodiment, the nonwoven web 56 and the formed film layer 58 are joined together such that the plurality of protuberances extend outwardly outwardly in an opposite direction of the nonwoven web 56 (see, for example, the composite sheet material shown in FIG. 2).

As in the previous embodiments, the apparatus 40a includes a film extrusion die 42 that extrudes a polymer melt curtain 44 onto a forming structure that rotates about a cylinder 48 having a vacuum slot 50 through which a vacuum is pulled. As the polymer melt curtain passes over the vacuum slot, the forming structure on the cylinder forms apertured protuberances comprising a plurality of raised projections extending outwardly from the film. The now formed film layer 58 is brought into contact with the nonwoven web 56 at roll 76. In the illustrated embodiment, a nonwoven web 56 is unwound from a supply roll 70.

In some embodiments, the formed film layer 58 having the apertured protuberances may still be in a molten or semi-molten state when brought into contact with the nonwoven web 56 so that the fibers of the nonwoven web adjacent to the film layer 58 embed into the surface of the film as the two layers pass between the pair of rollers 62, 60.

The resulting composite sheet 72a travels to one or more subsequent rollers until it may be wound onto a roll 68. It should be recognized that additional rollers and/or other pieces of equipment may be used in the apparatus 40 and any of the other apparatuses described herein for forming the composite sheet material.

In some embodiments, the apparatus 40a may include an optional bonding unit 64 to facilitate bonding of the film layer and nonwoven layer. In one embodiment, the bonding unit may comprise a calender bonding unit 65 comprising a pair of cooperating rolls 75a, 75b in which one of the rolls is heated to a temperature sufficient to bond the film layer to the fibers of the nonwoven web 56. In some embodiments, one of the rolls may be an anvil roll while the other roll has raised projections to facilitate point bonding of fabric, and to impart a desired pattern to the composite sheet material. Examples of suitable bonding patterns may include oval. cross-direction rod (CD rod), honeycomb, diamond, square, rectangular, circle, or the like.

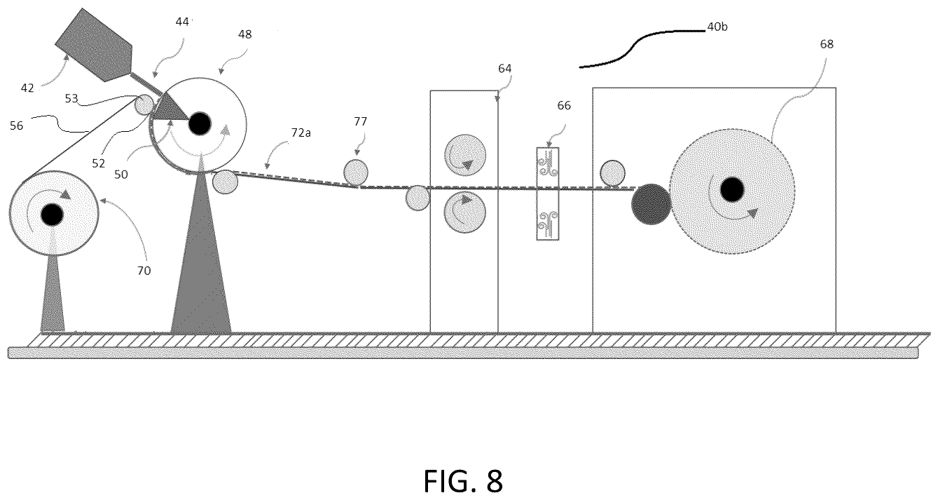

With reference to FIG. 8, a further apparatus 40b for preparing a composite sheet material in accordance with at least one embodiment is illustrated. In this embodiment, the apparatus 40b includes a film extrusion die 42 that extrudes a polymer melt curtain 44 onto a forming structure that rotates about a cylinder 48 having a vacuum slot 50 through which a vacuum is pulled. As the polymer melt curtain passes over the vacuum slot, the forming structure on the cylinder forms apertured protuberances comprising a plurality of raised projections extending outwardly from the film. In this embodiment, the nonwoven web 56 and the formed film layer 58 are joined together such that the plurality of protuberances extend outwardly in an opposite direction of the nonwoven web 56 (see, for example, the composite sheet material shown in FIG. 2).

Shortly, after the polymer melt curtain 44 is deposited onto the cylinder 48, the nonwoven web 56 is brought into contact with the still molten or semi molten formed film layer while the film is still on the surface of the cylinder 48. In certain embodiments, roll 53 feeds the nonwoven web 56 onto the surface of the cylinder overlying the formed film layer.

Preferably the formed film layer having the apertured protuberances is still in a molten or semi-molten state when brought into contact with the nonwoven web 56 so that the fibers of the nonwoven web adjacent to the formed film layer embed into the surface of the film to form the composite sheet material. In some embodiments, support rolls 73a, 73b may be present to help stabilize and support the composite sheet material.

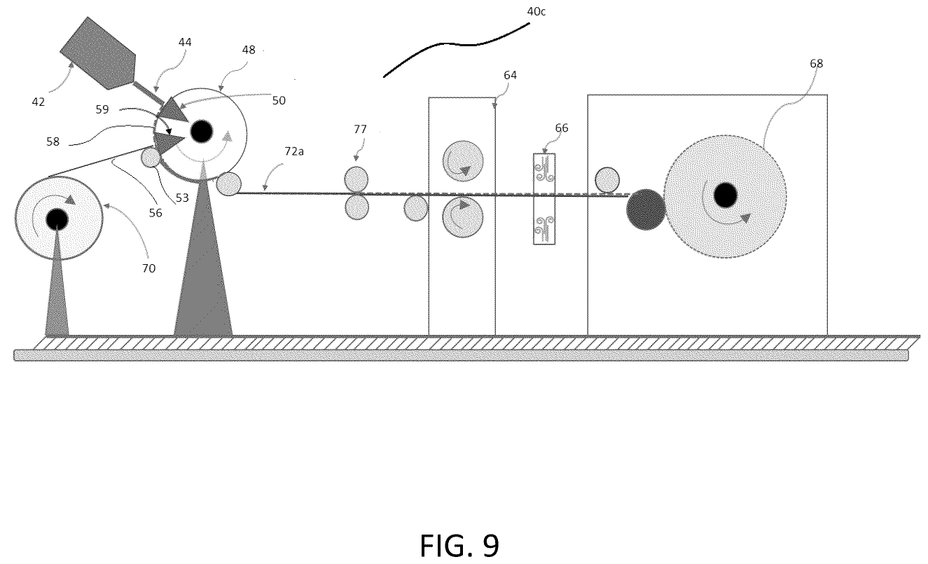

With reference to FIG. 9, yet a further embodiment of an apparatus 40c for preparing a composite sheet material in accordance with at least one embodiment is illustrated. In this embodiment, the apparatus 40c includes a film extrusion die 42 that extrudes a polymer melt curtain 44 onto a forming structure that rotates about a cylinder 48 having a vacuum slot 50 through which a vacuum is pulled. As the polymer melt curtain passes over the vacuum slot, the forming structure on the cylinder forms apertured protuberances comprising a plurality of raised projections extending outwardly from the film to form formed film layer 58. In this embodiment, the nonwoven web 56 and the film layer 58.

In this embodiment, the formed film layer 58 and carded nonwoven web 56 are joined together such that the plurality of protuberances extend outwardly in an opposite direction of the nonwoven web 56 (see, for example, the composite sheet material shown in FIG. 2).

Shortly, after the polymer melt curtain 44 is deposited onto the cylinder 48, the carded nonwoven web 56 is brought into contact with the still molten or semi molten film while the formed film is still on the surface of the cylinder 48. In certain embodiments, roll 53 feeds the carded nonwoven web 56 onto the surface of the cylinder overlying the formed film layer. A second slot 59 having an associated vacuum source is disposed on cylinder 48 downstream of vacuum slot 50. Second vacuum slot 59 helps facilitate pulling the fibers of the nonwoven web 56 into the still molten or semi-molten formed film layer to help facilitate bonding of the two layers.

In some embodiments, the system may include a pair of press rolls 77 to help facilitate lamination of the film layer and the nonwoven web.

Preferably the formed film layer having the apertured protuberances is still in a molten or semi-molten state when brought into contact with the nonwoven web 56 so that the fibers of the nonwoven web adjacent to the formed film layer embed into the surface of the film to form the composite sheet material.

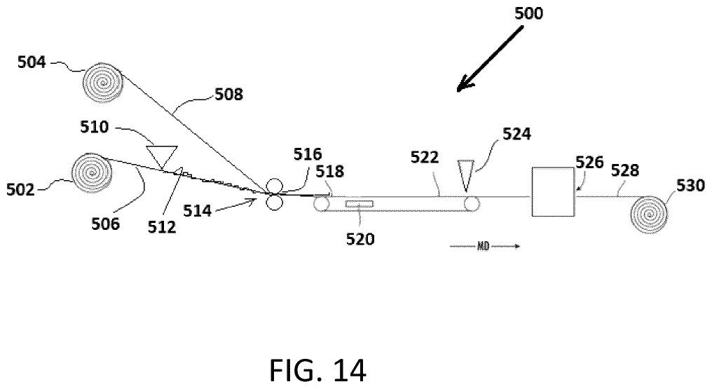

With reference to FIG. 14, a schematic illustration of a system for preparing a composite sheet material having at least one adhesive layer is shown and designated by reference character 500. The system includes a source of a nonwoven web 502 and a source of a microperforated film 504. In the illustrated embodiment, the source of the nonwoven web 502 is shown as a supply roll and the source of the microperforated film is also shown as a supply roll. It should be recognized that the source for the nonwoven web may comprise a spinbeam, such as a beam for preparing a spunbond nonwoven, or a system for preparing a carded nonwoven fabric. Similarly, the system may include an extrusion system and associated wire roll (see FIG. 5, reference characters 42 and 46) for preparing a microperforated film. In this way, the preparation of the film and microperforated film may be accomplished in a continuous in-line system. It should be understood that other nonwoven forming systems may be used in accordance with certain embodiments of the invention, such as spundond, meltblown, carded, air bonded, resin bonded, spunlace, and the like. In certain embodiments, the nonwoven web 506 comprises a high loft spunbond nonwoven (discussed previously).

In certain embodiments, the microperforated film layer comprises a plurality of protuberances that extend outwardly from the film layer. The plurality of protruberances may extend outwardly in an opposite direction of the nonwoven web 506 (see, for example, the composite sheet material shown in FIG. 2), or may extend in the direction of the nonwoven web see, for example, the composite sheet material shown in FIGS. 3A and 3B).

Nonwoven web 506 is unwound from supply roll 502 and microperforated film 508 is unwound from supply roll 504. A layer of adhesive is deposited onto a surface of the nonwoven web 506 via adhesive supply device 510. As noted previously, the adhesive may comprise one or more methods of forming a coating onto a surface of the nonwoven web, such as using known printing and coating techniques. Although not shown, the adhesive layer may optionally be applied to a surface of the microperforated film 508.

The nonwoven web 506 having the layer of adhesive and the microperforated film 508 are directed through a pair of cooperating rolls 514 (e.g., a pair of press rolls that help facilitate lamination of the film layer and the nonwoven web) that collectively define a nip 516. The nip applies pressure to adhesively bond the nonwoven web 506 and the microperforated film to each other to define a composite sheet material 518 having an adhesive layer.

Optionally, the composite sheet material 518 is directed onto a collection surface 522. An optional vacuum source 520 may be disposed below the surface of the collection surface 522.

In certain embodiments, an optional bonding unit 526 is disposed downstream of the collection surface 522 and is configured and arranged to thermally bond fibers to each other to form a coherent web. During thermal bonding the nonwoven web 506 comprising a plurality of fibers are heated to a temperature that is sufficient to soften at least one polymer component comprising fibers of nonwoven web 506 to produce a bonded nonwoven fabric 528. In certain embodiments, the composite sheet material 518 moves to a winder 530, where the composite sheet material is then wound onto rolls.

In certain embodiments, the bonding unit comprises an air through bonder in which the fibers are exposed to one or more streams of heated gas, such as air. In other embodiments, the bonding unit comprises a calender bonding unit. Both of these types of bonding units are discussed previously.

In some embodiments, an optional hot air knife 524 stabilizes the web of fibers by subjecting the fibers to a stream of heated gas, such as air, prior to delivery to the winder 530 or the bonding unit 526 for bonding. The hot air knife exposes the web composite sheet material 518 to a stream of heated gas to lightly bond and stabilize the web.

Composite Sheet materials in accordance with embodiments of the present invention may be used in a wide variety of applications. In particular, composite sheet materials previously discussed may be adapted for use in a disposable absorbent article such as a diaper, a pant, an adult incontinence product, a sanitary napkin or any other article that may benefit from the desirable properties provided with the composite sheet materials in accordance with embodiments of the present invention.

### Absorbent Articles

Composite sheets in accordance with the present invention may be used in a wide variety of different articles, and in particular, a wide variety of absorbent articles.

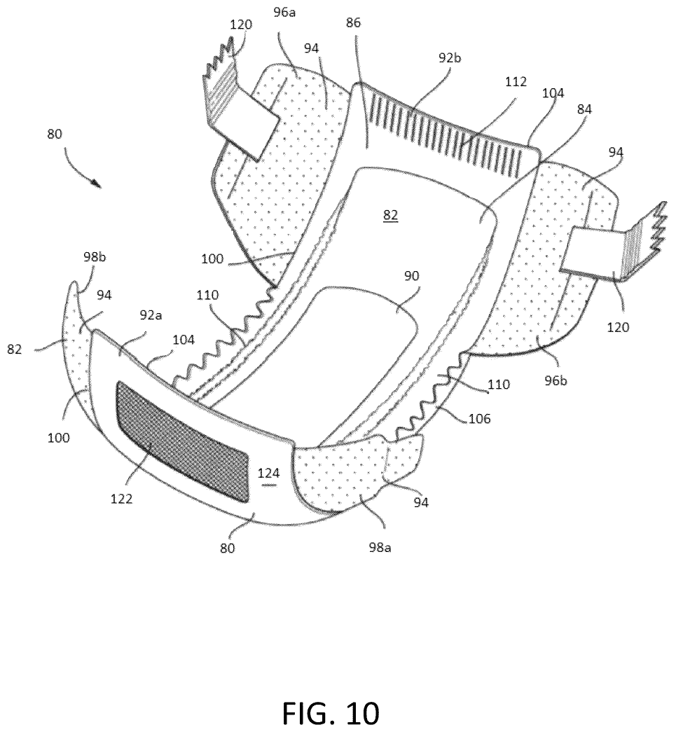

With reference to FIG. 10, an embodiment of an absorbent article (“diaper”) in accordance with embodiments of the present invention is shown and broadly designated by reference number 80. The diaper 80 includes a core region 82 in which an absorbent core 84 is disposed. A chassis region 86 surrounds the core region 82, and includes a front 88, back 90, and front and back waist regions 92a, 92b. The chassis region comprised of front, back and core regions generally has a composite structure comprising a liquid permeable topsheet and a liquid impermeable backsheet that are attached to each other along opposing surfaces to define a cavity there between in which the absorbent core is disposed.

Suitable materials for the topsheet, backsheet, and absorbent core may generally comprise any materials conventionally used in the manufacture of absorbent articles.

As shown in FIG. 10, the diaper also includes a composite sheet material 10 in accordance with at least one embodiment of the present invention. The composite sheet material 10 defines a fluid acquisition distribution layer 90 (i.e., ADL component) of the absorbent article. As discussed above, the composite sheet material 10 helps to efficiently facilitate transfer of fluid from the wearer to the absorbent core 84.

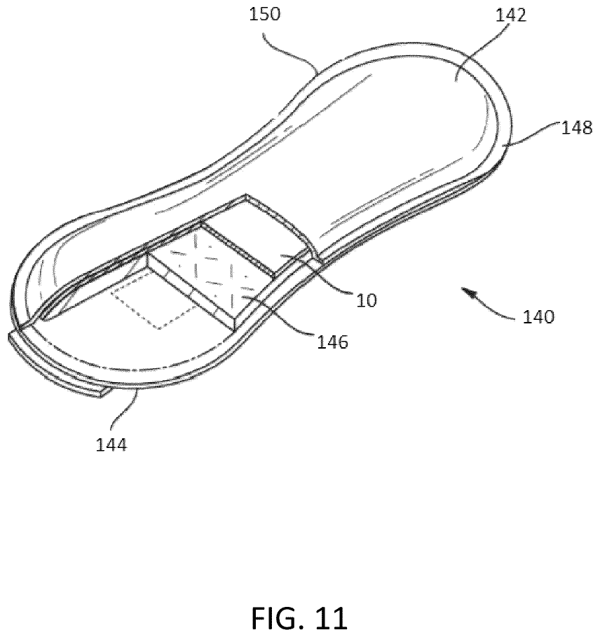

With reference to FIG. 11, a further embodiment of an absorbent article in accordance with an embodiment of the present invention is illustrated in which the absorbent article is in the form of a feminine sanitary pad, broadly designated by reference character 140.

Pad 140 may include a topsheet 142, backsheet 144, and an absorbent core 146 disposed there between. Preferably, topsheet 142 and backsheet 144 are joined to each other about along opposing outer edges to define a continuous seam 148 that extends about the periphery 150 of the pad 140. Continuous seam 148 may comprise a heat seal that is formed from thermally bonding the topsheet and backsheet to each other. In other embodiments, continuous seam 148 is formed by adhesively bonding the topsheet and backsheet to each other.

Suitable materials for the topsheet, backsheet, and absorbent core may comprise materials typically used in the construction of absorbent articles.

As shown, pad 140 includes a composite sheet 10 in accordance with embodiments of the invention defining a fluid ADL component 10. The ADL component is disposed between the absorbent core 146 and the topsheet 142. As discussed above, the composite sheet defining the ADL component comprises a perforated film layer and a nonwoven layer selected from the group consisting of a carded nonwoven and a high loft spunbond nonwoven.

Various components of the absorbent article are typically joined via thermal or adhesive bonding. Examples of suitable adhesives include polyethylene, polypropylene, or ethylene vinyl acetate based melt adhesives. In some embodiments, the adhesive may comprise a bio-based adhesive. An example of a bio-based adhesive is a pressure sensitive adhesive available from Danimer Scientific under the product code 92721.

In yet another aspect, certain embodiments of the invention provide absorbent articles. In accordance with certain embodiments, the absorbent article may include a composite sheet in accordance with the present invention.

In this regard, composite sheets prepared in accordance with embodiments of the invention may be used in wide variety of articles and applications. For instance, embodiments of the invention may be used for personal care applications, for example products for babycare (diapers, wipes), for femcare (pads, sanitary towels, tampons), for adult care (incontinence products), or for cosmetic applications (pads).

In certain embodiments, the inventive nonwoven fabric may be combined with one or more additional nonwoven layers to prepare a composite or laminate material.

Examples of such composites/laminates may include a spunbond composite, such as a spunbond-meltblown (SM) composite, a spunbond-meltblown-spunbond (SMS) composite, or a spunbond-meltblown-meltblown-spunbond (SMMS) composite), a spunbond-spunbond-meltblown-meltblown-spunbond (SSMMS), or a spunbond-spunbond-meltblown-spunbond (SSMS) composite. In some embodiments, composites may be prepared comprising a layer of the bonded nonwoven fabric and one or more film layers. It should be recognized other configurations are also in the scope of the invention.
comprise the same spunbond nonwoven fabric layer, or two different spundbond layers.

In these multilayer structures, the basis weight of the spunbond nonwoven fabric layers may range from as low as 5 g/m² and up to 200 g/m². In such multilayered laminates, both the meltblown and spunbond fibers could have a similar polymer on the surface of the fibers to help facilitate optimum bonding. In some embodiments in which the inventive meltblown layer is a part of a multilayer structure (e.g., SM, SMS, and SMMS), the amount of the meltblown in the structure may range from about 10 to 90%, and in particular, from about 40 to 75% of the structure as a percentage of the structure as a whole.

Multilayer structures in accordance with embodiments can be prepared in a variety of manners including continuous in-line processes where each layer is prepared in successive order on the same line, or depositing a second nonwoven layer, such as a meltblown layer, on a previously formed spunbond layer. The layers of the multilayer structure can be bonded together to form a multilayer composite sheet material using thermal bonding, mechanical bonding, adhesive bonding, hydroentangling, or combinations of these.

The following examples are provided for illustrating one or more embodiments of the present invention and should not be construed as limiting the invention.

### EXAMPLES

In the following examples described in Tables 1 and 2, a simulated composite sheet material in accordance with embodiments of the invention were evaluated for fluid management properties in commercially available diapers: Target brand UP&UP™, and HUGGIES® Little Snugglers™. In these examples, the simulated composite sheet comprised a through air bonded carded nonwoven and a perforated film layer overlying the carded nonwoven.

In the evaluation, the commercially obtained diapers (Size 4) were opened up along the top sheet and the existing ADL layer was removed and replaced with a simulated composite sheet material consisting of a microperforated film layer and a carded through air bonded nonwoven fabric layer. In these evaluations, the microperforated film layer and the carded through air bonded nonwoven fabric layer were not bonded to each other.

The simulated composite sheet material was oriented so that the microperforated film layer was disposed underneath the diaper topsheet with the apertured protuberances extending towards the top sheet. The carded through air bonded nonwoven fabric layer was positioned underlying the microperforated film layer (i.e., between the microperforated film layer and the absorbent core).

“MF-FILM” refers to a perforated polyethylene cast extruded film having a basis weight of 24 gsm.

The following carded nonwoven fabrics were used:

“ATB-1” refers to a carded through air bonded fabric having a basis weight of 40 gsm.

“ATB-2” refers to a carded through air bonded fabric having a basis weight of 30 gsm.

“ATB-3” refers to a carded through air bonded fabric having a basis weight of 44 gsm

The samples were then evaluated for fluid acquisition time and fluid rewet.

### Procedure for Fluid Acquisition Time (NWSP 0.70.9.R1 (15) Test Method)

80 mL of 0.9% saline solution was measure into a separatory funnel. The solution was dyed to make it easier to determine when the solution was fully absorbed. A 5.75 lb. annular dosing weight was used. The outer and inner diameters of the dosing weight were 4 and 1 inches, respectively. The dosing weight was placed on a central portion of the diaper overlying the diaper's topsheet. The saline solution was then poured into the inner diameter of the dosing weight. A stop watch was started as soon as the solution came in contact with the surface of the diaper. Once all the fluid had been absorbed, the stop watch was stopped and the fluid acquisition time was recorded.

### Procedure for Fluid Rewet

Following the fluid acquisition time evaluation, the dosing weight was allowed to remain on the diaper for 10 minutes. The dosing weight was then removed and a stack of 20-filter papers (weight previously measured) were placed overlying the topsheet. A simulated baby weight (10 cm×10 cm block weight 4.00 kg) was then placed on the stack of filter papers for 2 minutes. The weight was then removed and the weight of the filter papers was measured. The dry weight of the filter papers was then subtracted from the wet weight to provide a rewet weight. For fluid acquisition times and rewet values for additional fluid insults, the above steps were repeated using the same diaper.

TABLE 1
Evaluation of Composite Sheet in Target Brand UP&UP

| | | Average Acquisition | | | | | |
|---|---|---|---|---|---|---|---|
| | | Time (s) | | | Average of Rewet (g) | | |
| Sample | | Insult | Insult | Insult | Insult | Insult | Insult |
| No. | ADL | 1 | 2 | 3 | 1 | 2 | 3 |
| Sample 1 | ATB-1 | 34.67 | 35.00 | 38.00 | 0.5270 | 0.4897 | 0.5530 |

TABLE 2
Evaluation of Composite Sheet in
HUGGIES Brand LITTLE SNUGGLERS

| | | Average Acquisition | | | | | |
|---|---|---|---|---|---|---|---|
| | | Time (s) | | | Average of Rewet (g) | | |
| Sample | | Insult | Insult | Insult | Insult | Insult | Insult |
| No. | ADL | 1 | 2 | 3 | 1 | 2 | 3 |
| Sample 2 | ATB-2 | 47.00 | 64.67 | 76.00 | 0.417 | 0.437 | 0.784 |
| Sample 3 | ATB-3 | 50.67 | 57.33 | 65.00 | 0.383 | 0.426 | 1.877 |

Evaluation of Composite Sheet comprising a high loft nonwoven and a perforated film layer.

In the following examples of Tables 3 and 4, two layer composite sheets comprising a high loft spunbond layer and a perforated film layer were prepared and evaluated for various properties including fluid handling properties.

The microperforated film layer comprised a polymeric blend of 28.5 weight percent low density polyethylene (available from Chevron under the product code 4571), 59.5 weight percent of high density polyethylene (available from Dow under the product code 9607), 5 weight percent of whitener masterbatch (available from AMPACET under the product code A11399-E), and 7 weight percent of a surfactant (available from AMPACET under the product code A11399-A1001153-NP).

The nonwoven substrate of Samples 4-5 comprised a through air bonded spunbond nonwoven fabric composed of bicomponent filaments having a sheath/core configuration in which the core to sheath ratio was 60:40. The sheath and the core both comprised polypropylene (available from Borealis). The nonwoven substrate of Samples 4 and 5 had a basis weight of 20 gsm.

The film was extruded under vacuum onto a micorperforation forming surface and then, while still in a semi-molten state, deposited onto a surface of the nonwoven substrate to form the composite sheet material. The film was oriented such that the apertured protuberances extended outwardly away from the nonwoven substrate. An aqueous solution comprising 12 percent by volume of a hydrophilic finish agent (available from Schill+Selakcher under the product name Silastol 163) was applied to the surface of the film layer and then dried.

The resulting composite sheets were evaluated. The results are provided in Table 3, below.

The following test methods were used to evaluate Samples 4-5.
Basis Weight: ASTM D 646.
Embossed Thickness: ASTM D 645-97.
Air Permeability: ASTM D 737.
Strikethrough/Rewet: EDANA 150.5-02 and 151.3.02.

TABLE 3
Average Properties of Samples 4-5

| | | Air | | | | |
|---|---|---|---|---|---|---|
| | | Perme- | | | | |
| | Embossed | ability | Basis | Strike | | Peel |
| Sample | Thickness | (m³/m²/ | weight | through | Rewet | Bond |
| Number | (mm) | min.) | (gsm) | (s) | (g) | (g) |
| Sample 4 | 0.81 | 82 | 41.13 | 0.43 | 0.07 | 170 |
| Sample 5 | 1.08 | 105 | 60.21 | 0.43 | 0.06 | 404 |

Values based on average of 3 tests per sample.

That which is claimed:

1. A composite sheet material comprising
a nonwoven substrate selected from the group consisting of a carded nonwoven and a high loft spunbond nonwoven, wherein the carded nonwoven comprises a plurality of staple fibers bonded to each other to form a coherent web, and has a basis weight from about 16 to 80 gsm, and the high loft spunbond nonwoven has a density of less than 0.080 g/cm³; and
a microperforated polymeric film layer overlying and bonded to the nonwoven substrate, wherein the film comprises a plurality of apertured protuberances extending outwardly from a face of the film, the apertures including a first opening disposed opposite the nonwoven substrate, a second opening at a vertex of the aperture, and a continuous sidewall extending between the first and second openings.

2. The composite sheet material according to claim 1, wherein the plurality of apertured protuberances extend outwardly from a face of the film towards a surface of the nonwoven substrate.

3. The composite sheet material according to claim 1, wherein the plurality of apertured protuberances extend outwardly from a face of the film away from a surface of the nonwoven substrate.

4. The composite sheet material according to claim 1, wherein the nonwoven substrate comprises a through air bonded fabric, a high loft spunbond nonwoven, or a a resin-bonded nonwoven.

5. The composite sheet material according to claim 1, further comprising an adhesive layer disposed between the nonwoven substrate and the film layer.

6. The composite sheet material according to claim 1, wherein the polymer defining the film layer is contacted with a surface of the nonwoven substrate in a molten or semi-molten state such that the polymer is at least partially integrated into and between the fibers of the nonwoven substrate to form a mechanical bond between the nonwoven substrate and the film layer.

7. The composite sheet material according to claim 1, wherein the plurality of apertured protuberances have a conical shape.

8. The composite sheet material according to claim 1, wherein the film comprises polyethylene.

9. The composite sheet material according to claim 1, wherein the nonwoven substrate comprises a carded nonwoven fabric layer having a basis weight from about 30 to 50 gsm, or the nonwoven substrate comprises a high loft spunbond selected from the group consisting of a through air bonded nonwoven, a spunbond nonwoven comprising a plurality of crimped filaments; a spunbond nonwoven in which fibers of the nonwoven comprise a blend of a polymer and a high loft additive, and combinations thereof.

10. The composite sheet material according to claim 1, wherein the high loft spunbond nonwoven comprises a high loft additive, the high loft additive comprising an aliphatic fatty acid amide having at least one amide group and at least one aliphatic carbon chain having from 10 to 22 carbon atoms.

11. The composite sheet material according to claim 10, wherein the high loft additive includes fatty acid amides of the general formula (1): 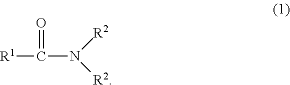
where R¹ comprises a C₁₀-C₂₂ carbon chain, which may be saturated or unsaturated and aliphatic, and R², independently, is selected from H, a C₁-C₄ alkyl group, R¹, and R³, with R³ having the following formula (2):
—(C₁-C₄)C(═O)N(R⁵)(R¹) (2)
wherein R⁵ is H or a C₁-C₄ alkyl group, and R⁵ is preferably H.

12. The composite sheet material according to claim 1, wherein the composite sheet material exhibits:
an average fluid acquisition times of less than 3 seconds after a first fluid insult and less than 5 seconds after a second fluid insult; and
a fluid rewet value following a single fluid insult ranging from about 0.05 to 1.0 g, and in particular, from about 0.06 to 0.08 g.

13. The composite sheet material according to claim 1, wherein the composite sheet material exhibits a fluid acquisition time after a first fluid insult of less than 1 seconds and a rewet of less than 0.15 grams.

14. A method of preparing a composite sheet material, the method comprising the steps of
providing a nonwoven substrate, the nonwoven substrate selected from the group consisting of a carded nonwoven fabric and a high loft spunbond nonwoven;
extruding a polymer film onto a cylinder having a forming surface thereon while the polymer film is in a molten or semi-molten state.
forming a plurality of apertured protuberances in the film to form a formed film layer, wherein each apertured protuberance including a proximal end, a distal end, and a continuous sidewall extending therebetween;
contacting a surface of the nonwoven substrate to a surface of the formed film layer; and
bonding the nonwoven substrate to the formed film to form a composite sheet material.

15. The method of claim 14, wherein the step of contacting the nonwoven substrate and the formed film layer comprises a step of introducing the nonwoven substrate onto the formed film layer while the formed film layer is still on the surface of said cylinder.

16. The method of claim 14, wherein the step of contacting the nonwoven substrate and the formed film layer is performed after a step of drawing the formed film layer off said cylinder.

17. The method according to claim 14, wherein the polymer defining the film layer is contacted with a surface of the nonwoven substrate in a molten or semi-molten state such that the polymer is at least partially integrated into and between fibers of the nonwoven substrate to form a mechanical bond between the nonwoven substrate and the formed film layer.

18. The method according to claim 14, wherein the cylinder includes a slot extending laterally across its length, and wherein a vacuum is applied through the slot to the film layer overlying the slot.

19. The method according to claim 14, wherein the nonwoven substrate comprises a carded nonwoven comprising a plurality of staple fibers bonded to each other to form a coherent web, the carded nonwoven fabric having a basis weight from about 16 to 80 gsm, or the nonwoven substrate comprises a high loft spunbond nonwoven having a density of less than 0.080 g/cm³.

20. The method of claim 19, wherein the high loft spunbond nonwoven is selected from the group consisting of a through air bonded nonwoven, a spunbond nonwoven comprising a plurality of crimped filaments; a spunbond nonwoven in which fibers of the nonwoven comprise a blend of a polymer and a high loft additive, and combinations thereof.

21. A method of preparing a composite sheet material, the method comprising the steps of
providing a nonwoven substrate, the nonwoven substrate selected from the group consisting of a carded nonwoven fabric and a high loft spunbond nonwoven;
providing a polymer film comprising plurality of apertured protuberances in the film, wherein each apertured protuberance including a proximal end, a distal end, and a continuous sidewall extending therebetween;
depositing a layer of adhesive on one or more of a surface of the nonwoven substrate or a surface of the polymer film such that the adhesive layer is disposed between the nonwoven substrate and the polymer film; and
contacting a surface of the nonwoven substrate to a surface of the formed film layer; and adhesively bonding the nonwoven substrate to the formed film to form a composite sheet material.
